(19) United States
(12) Patent Application Publication
(10) Pub. No.: US 2016/0340324 A1
(43) Pub. Date: Nov. 24, 2016

(54) METHODS AND COMPOSITIONS FOR TREATING CANCER

(71) Applicants: Ying Su, La Jolla, CA (US); Sanford-Burnham Medical Research Institute, La Jolla, CA (US)

(72) Inventors: Ying SU, La Jolla, CA (US); Xiao-kun ZHANG, La Jolla, CA (US)

(21) Appl. No.: 15/107,275

(22) PCT Filed: Dec. 22, 2014
(86) PCT No.: PCT/US2014/071989

§ 371 Date: Jun. 22, 2016

Related U.S. Application Data
(60) Provisional application No. 61/920,264, filed on Dec. 23, 2013.

(51) Int. Cl. C07D 257/04; G06N 7/00; G06F 19/00; A61K 31/41; A61K 38/19

(52) U.S. Cl. CPC C07D 257/04; A61K 31/41; A61K 38/191; G06F 19/704; G06F 19/706; G06N 7/005

(57) ABSTRACT

Embodiments provided herein relate to methods and compositions for treating cancer. Some embodiments relate to certain compounds having activity against retinoid X receptor-alpha (RXRα). Some embodiments included designing or identifying a compound that binds to human RXRa protein, such as the ligand binding domain (LBD) of human RXRa protein.

### CROSS-REFERENCE TO RELATED APPLICATIONS

This application claims the benefit of U.S. Provisional Application No. 61/920,264 filed Dec. 23, 2013 entitled “SULINDAC-DERIVED RXR-ALPHA MODULATORS AND USES THEREOF” which is incorporated herein by reference in its entirety.

### STATEMENT REGARDING FEDERALLY SPONSORED R&D

This invention was made with government support under W81XWH-11-1-0677 awarded by the U.S. Army Medical Research and Material Command, and CA140980, GM089927, and CA179379 awarded by the National Institutes of Health. The government has certain rights in the invention.

### FIELD OF THE INVENTION

Embodiments provided herein relate to methods and compositions for treating cancer. Some embodiments relate to certain compounds having activity against retinoid X receptor-alpha (RXRα). Some embodiments included designing or identifying a compound that binds to human RXRα protein, such as the ligand binding domain (LBD) of human RXRα protein.

### BACKGROUND OF THE INVENTION

Retinoid X receptor alpha (RXRα), a unique member of the nuclear receptor superfamily, regulates a broad spectrum of physiological functions including cell differentiation, growth and apoptosis (Germain et al., 2006; Szanto et al., 2004). Like other nuclear receptors, RXRα acts as a ligand-dependent transcription factor (Germain et al., 2006; Szanto et al., 2004). RXRα may have extranuclear actions. RXRα resides in the cytoplasm at certain stages during development (Dufour and Kim, 1999; Fukunaka et al., 2001) and migrates from the nucleus to the cytoplasm in response to differentiation, apoptosis, and inflammation (Cao et al., 2004; Casas et al., 2003; Zimmerman et al., 2006). RXRα

RXRα exhibits a modular organization structurally consisting of three main functional domains: an N-terminal region, a DNA-binding domain and a ligand-binding domain (LBD). The LBD possesses a ligand-binding pocket (LBP) for the binding of small molecule ligands, a transactivation function domain termed AF-2 composed of Helix 12 (HI2) of the LBD, a coregulator binding surface, and a dimerization surface (Germain et al., 2006; Szanto et al., 2004). The ligand-dependent transcription regulation is predominately mediated through H12 that is highly mobile. Agonist ligand binds to the LBP and helps the H12 to adopt the active conformation that forms a surface to facilitate the binding of coactivators and subsequent transactivation. In contrast, in the absence of an agonist ligand or in the presence of an antagonist ligand, the H12 adopt an inactive conformation that favors the binding of corepressors to inhibit target gene transcription. Natural RXRα ligand 9-cis-retinoic acid (9-cis-RA) and synthetic ligands have been effective in preventing tumorigenesis in animals and RXRα has been a drug target for therapeutic applications, especially in the treatment of cancer (Bushue and Wan, 2010; Yen and Lamph, 2006). RXRα can bind to DNA and activate transcription of target genes either as a homodimer or a heterodimer with its heterodimerization partners including retinoic acid receptor (RAR), vitamin D receptor (VDR), thyroid hormone receptor (TR), and peroxisome-proliferator-activated receptor (Germain et al., 2006; Szanto et al., 2004). In addition to homodimer and heterodimer, RXRα could also self-associate into homotetramers in solution, which rapidly dissociate into active dimers upon binding of a cognate ligand (Chen et al., 1998; Kersten et al., 1995). Tetramer formation of RXRα might serve to sequester the receptor's active species, dimers and monomers, into a transcriptionally inactive tetramer complex (Gampe et al., 2000).

Efforts on discovery of small molecules targeting RXRα for therapeutic application have been primarily focused on the optimization of the molecules that bind to its classical LBP (de Lera et al., 2007; Germain et al., 2006; Szanto et al., 2004). However, various studies have recently identified small molecule modulators of nuclear receptors that function via unknown sites and undefined mechanisms of action (Buzon et al., 2012; Moore et al., 2010). Compounds that bind to RXRα at the sites other than the classical LBP have not been reported.

### SUMMARY OF THE INVENTION

Some embodiments of the methods and compositions provided herein include a compound of Formula (I) having the structure:

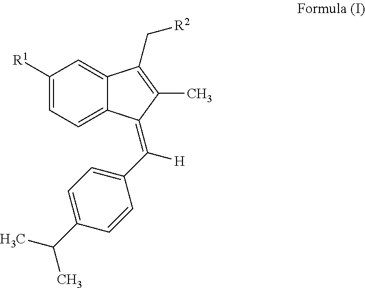

or tautomers thereof, wherein:

R¹ is H or halogen; and

R is C(═O)OH or

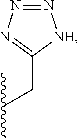 [SMILES: *Cc1nnn[nH]1]

In some embodiments, R¹ is H.

In some embodiments, R¹ is F.

In some embodiments, R² is C(═O)OH.

In some embodiments, R² is

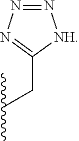 [SMILES: *Cc1nnn[nH]1]

In some embodiments, the compound has the structure:

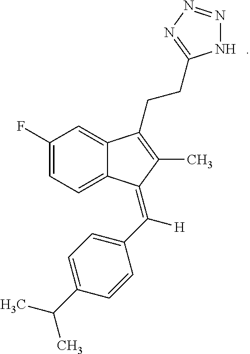 [SMILES: CC1=C(CCc2nnn[nH]2)c2cc(F)ccc2/C1=C\c1ccc(C(C)C)cc1]

In some embodiments, the compound has structure:

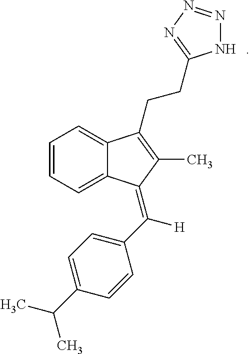 [SMILES: CC1=C(CCc2nnn[nH]2)c2ccccc2/C1=C\c1ccc(C(C)C)cc1]

Some embodiments of the methods and compositions provided herein include a pharmaceutical composition comprising any one of the foregoing compounds and a pharmaceutically acceptable excipient. Some embodiments of the methods and compositions provided herein include method of inhibiting a tumor cell comprising contacting the tumor cell with any one of the foregoing compounds. Some embodiments also include contacting the tumor cell with Tumor necrosis factor (TNFα).

In some embodiments, the tumor cell is selected from the group consisting of a lung tumor cell, a prostate tumor cell, a breast tumor cell, a colon tumor cell, a colon tumor cell, a liver tumor cell, and a lung tumor cell. In some embodiments, herein the tumor cell is selected from the group consisting of PC3, ZR-75-1, HeLa, HCT-116, A549, MB231, HepG2, and CV-1. In some embodiments, the tumor cell is in vivo. In some embodiments, the tumor cell is in vitro. In some embodiments, the tumor cell is mammalian. In some embodiments, the tumor cell is human.

Some embodiments of the methods and compositions provided herein include a method of treating a tumor comprising administering to a subject in need thereof an effective amount of the foregoing pharmaceutical compositions. Some embodiments also include administering an effective amount of Tumor necrosis factor (TNFα) to the subject. In some embodiments, the cancer is liver cancer. In some embodiments, the subject is mammalian. In some embodiments, the subject is human.

Some embodiments of the methods and compositions provided herein include a kit comprising any one of the foregoing compounds and a pharmaceutically acceptable excipient. Some embodiments also include an additional therapeutic agent. In some embodiments, the additional therapeutic agent comprises Tumor necrosis factor (TNFα).

Some embodiments of the methods and compositions provided herein include a method of designing a compound that binds to human RXRα protein comprising: accessing data comprising the structure of at least the ligand binding domain (LBD) of human RXRα protein; and modeling the binding of the compound to human RXRα protein using said data.

Some embodiments of the methods and compositions provided herein include a method for identifying a compound for inhibiting a tumor cell comprising the foregoing method.

In some embodiments, the modeling further comprises predicting the likelihood that the compound binds to a hydrophobic region of the LBD that does not overlap with the binding site of 9-cis-retinoic acid. Some embodiments also include predicting that the compound does not change the conformation of the LBP. In some embodiments, the compound is predicted to bind to a region of the LBD comprising at least one residue selected from the group consisting of Ala271, Ala272. Trp305, Leu309, Leu326, Leu330, Leu433, Leu436, Phe437, Phe438, Ile442, Gly443, and Leu436. In some embodiments, the compound is designed de novo. In some embodiments, the compound is designed from a known chemical entity of fragment thereof. In some embodiments, the chemical entity is any one of the foregoing compounds.

In some embodiments, the chemical entity is:

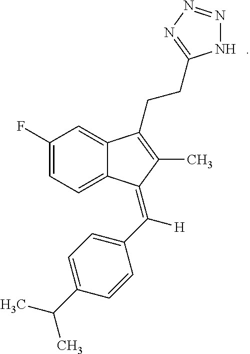 [SMILES: CC1=C(CCc2nnn[nH]2)c2cc(F)ccc2/C1=C\c1ccc(C(C)C)cc1]

In some embodiments, the chemical entity is:

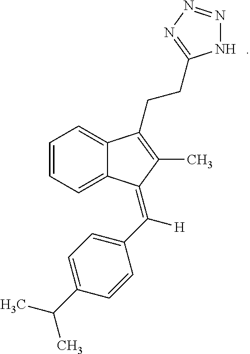 [SMILES: CC1=C(CCc2nnn[nH]2)c2ccccc2/C1=C\c1ccc(C(C)C)cc1]

### BRIEF DESCRIPTION OF THE DRAWINGS

FIG. 1A depicts the structures of Sulindac, compounds K-80003, K-8008, and K-8012. FIG. 1B depicts a synthesis scheme for compounds K-8008 and K-8012.

FIGS. 2A and 2B are graphs of relative CAT activity. (TREpal)₂-tk-CAT and RXRα were transiently transfected into CV-1 cells. Cells were treated with or without 9-cis-RA (10⁻⁷ M) in the presence or absence of the indicated concentration of Sulindac or other compounds. CAT activity was determined. BI-1003 (1 μM) was used for comparison. Error bars represent SEM. FIG. 2C is a graph of relative LUC activity. pBind-RXRα-LBD and pG5luc were transiently transfected into HCT-116 cells. Cells were treated with or without 9-cis-RA (10⁻⁷ M) in the presence or absence of BI-1003 (1 μM), K-8008 (50 μM) and K-8012 (50 μM). Luciferase activity was determined. FIG. 2D is a graph of relative LUC activity, depicting dose dependent effect of K-8008 and K-8012. HCT-116 cells transfected with pBind-RXRα-LBD and pG5luc were treated with indicated concentrations of K-8008 and K-8012 in the presence or absence of 9-cis-RA (10⁻⁷ M). Compounds 1, 2, 3, and 4 are sulindac, K-80003, K-8003, and K-8012, respectively.

FIGS. 3A-3H depict biological effects of K-8008 and K-8012. Compounds 1, 2, 3, and 4 are sulindac, K-80003, K-8003, and K-8012, respectively. FIG. 3A depicts growth inhibition by Sulindac and other compounds. A549 lung cancer cells were treated with various concentrations of the indicated compounds. Cell viability was measured by the MTT colorimetric assay. FIG. 3B depicts inhibition of TNFα-induced AKT activation. A549 cells were pretreated with Sulindac or other compounds for 1 h before exposed to TNFα (10 ng/mL) for 30 min. Phosphorylated AKT and total AKT were analyzed by immunoblotting. FIG. 3C depicts induction of apoptosis by Sulindac or other compounds in the presence of TNFα. Cells cultured in medium with 1% FBS were treated with TNFα (10 ng/mL) and/or compound (40 μM) for 4 h, and analyzed for PARP cleavage by immunoblotting. FIG. 3D depicts RXRα siRNA transfection inhibits the apoptotic effect of K-8008. HeLa cells transfected with control or RXRα siRNA for 48 h were treated with K-8008 (40 μM) and/or TNFα (10 ng/ml) for 6 h and analyzed by immunoblotting. FIG. 3E depicts RXRα siRNA transfection antagonizes the inhibitory effect of K-8008 on AKT activation. HeLa cells transfected with control or RXRα siRNA for 48 h were treated with K-8008 (40 μM) for 1.5 h before exposed to TNFα (10 ng/ml) for 30 min and analyzed by immunoblotting. FIG. 3F depicts Myc-RXRα-A80 transfection enhances the apoptotic effect of K-8008. HeLa cells transfected with Myc-RXRα or Myc-RXRα-A80 were treated with K-8008 (20 μM) and/or TNFα for 12 h. PARP cleavage and transfected RXRα expression were analyzed by immunoblotting. FIG. 3G depicts K-8008 inhibits the interaction of transfected tRXRα and p85α. HeLa cells transfected with Myc-RXRα-A80 and/or Flag-p85α for 24 h were treated with vehicle or 20 μM K-8008 in the presence of absence of 10 ng/ml TNFα for 1 h. Cell lysates were immunoprecipitated using anti-Myc antibody and analyzed by Western blotting (WB) analysis using the indicated antibody. FIG. 3H depicts K-8008 inhibits the interaction of endogenous tRXRα and p85α. A549 cells were pretreated with vehicle or 40 μM K-8008 for 1 h before exposed to 10 ng/mL TNFα for 30 min. Cell lysates were immunoprecipitated with AN 197 anti-RXRα antibody and analyzed by Western blotting.

FIGS. 4A-4F depict inhibition of HepG2 tumor growth in animals. FIG. 4A is a photograph of tumors. FIG. 4B is a graph of tumor volume. FIG. 4C is a graph of tumor weight. FIGS. 4A, 4B and 4C depict nude mice with HepG2 heptoma xenografts were intraperitoneally injected daily with vehicle, K-8008 (20 mg/kg) or K-80003 (20 mg/kg) for 12 days. Tumors were removed and measured. Tumor sizes and weights in control, K-80003 and K-8008-treated mice were compared. FIG. 4D is a Western blot. Lysates prepared from three tumors treated with vehicle or K-8008 were analyzed by Western blotting assay for p-AKT expression. FIG. 4E is a series of photomicrographs. H&E staining and TUNEL assay. Tumor sections were stained for H&E or TUNEL by immunohistochemistry. Increased apoptotic tumor cells were observed in tumor from K-8008 treated mice. FIG. 4F is a graph of body weight. K-8008 does not exhibit apparent toxicity. Body weight was measured every three days. Each point represents the mean±standard deviation of six mice. The differences between the compound treated group and control group are not significant (P>5%).

FIG. 5A is a graph of % bound 9-cis-RA. K-8008 and K-8012 fail to compete with the binding of 9-m-RA to RXRα. RXRα-LBD protein was incubated with [3H]9-cis-RA in the presence or absence of K-80003, K-8008, K-8012, or unlabeled 9-cis-RA. Bound [³H]9-cis-RA was quantitated by liquid scintillation counting. FIG. 5B is a TR-FRET graph. K-8008 and K-8012 reduce 9-cis-RA-induced FRET signal. GST-RXRα-LBD was incubated with K-8008 or K-8012 in the presence or absence of 9-cis-RA (10⁻⁷ M). B1-1003 (1 μM) was used as a control. FIG. 5C is a graph of TR-FRET and depicts dose dependent effect of K-8008 and K-8012 on 9-cis-RA-induced FRET signal. GST-RXRα-LBD was incubated with K-8008 or K-8012 in the presence of 9-cis-RA (10⁻⁸ M). Compounds 1, 2, 3, and 4 are sulindac, K-80003, K-8003, and K-8012, respectively

FIG. 6A depicts the tetramer structure of RXRα LBD in complex with K-8008. The two bound K-8008 molecules are shown as sticks surrounded by an electron density mesh. The two biological dimers (A1-B1 and A2-B2) are shown as pairs. The N- and C-termini of four subunits are marked by the corresponding residue numbers. FIG. 6B depicts superposition of the RXRα LBD monomers from the K-8008-binding structure and the apo protein structure (from PDB 1G1U). K-8008 is shown as sticks. The classic ligand binding site is also marked by a VDW ball model of 9-cis-RA taken from a superimposed PDB entry 1FBY. FIG. 6C depicts the hydrophobicity of the K-8008 binding site presented as a surface fragment on top of the RXRα LBD monomer. The hydrophobic side chains that contribute to the region are shown in teal and K-8008 is shown in the same fashion as in FIG. 6B. For clarity, residues contributing to this region are not labeled. FIG. 6D depicts the protein side chains (in sticks) that make VDW interaction with K-8008. The displayed region is an enlargement of the black box in FIG. 6A. The view is slightly rotated, and fragments of the green subunit that do not interact with K-8008 are removed. The protein surface is shown as semitransparent envelope. The (Fo-Fc) electron density is shown around the ligands as a black mesh. It was calculated at a 3-σ level with omitted ligand atoms. The positive end of the H11 helix dipole is highlighted in orange. FIG. 6E depicts side chains around K-8008 that make significant changes in comparison with the apo protein (PDB 1G1U). K-8008 is presented in the same fashion as in FIG. 6B.

FIGS. 7A-D are a structural comparison and mutagenesis Analysis of K-8008. FIG. 7A is a structural superposition of the protein/K-8008 complex and the protein/9-cis-RA complex. The 9-cis-RA-bound structure (PDB code 1FBY) is in pink cartoon, and 9-cis-RA is in cyan (C atoms) and red (O atoms) sticks. The K-8008-bound structure is in light orange cartoon, and K-8008 is in gray (C atoms) and blue (N atoms) sticks. Side chain Arg316 is displayed for distance comparison between distances to —COOH and to tetrazole. FIGS. 7B-7D are a mutational analysis of the K-8008 binding site. The LBD of RXRα or mutants cloned into pBind vector and pG5luc were transiently cotransfected into HCT-116 cells. Cells were treated with or without 9-cis-RA (10_7 M) in the presence or absence of BI-1003 (1 mM) or K-8008 (50 mM). Luciferase (LUC) activity was determined.

FIGS. 8A-C are a series of graphs depicting that sulindac and other compounds induce apoptosis of cancer cells. FIG. 8A is a PC3 prostate cancer cell line. FIG. 8B is a ZR-75-1 breast cancer cell line, and FIG. 8C is a MDA-MB-231 breast cancer cell line. Each were treated with the indicated concentration of Sulindac or other compounds for 48 h. Cell viability was determined by the MTT assay.

FIGS. 9A-D depict a series of Western blots depicting Sulindac and other compounds inhibit TNFα-induced AKT activation. The indicated cell lines were pretreated with indicated compounds for 1 h before exposed to TNFα (10 ng/mL) for 30 min. AKT activation and total AKT expression were analyzed by immunoblotting.

FIGS. 10A-10C depict induction of apoptosis by Sulindac and other compounds. FIG. 10A depicts the apoptotic effects of Sulindac or other compounds in different cell lines. PC3 human prostate cancer cells and HCT-116 human colon cancer cells were treated with 40 μM indicated compound for 6 h. PARP cleavage was analyzed by immunoblotting. FIG. 10B depicts synergistic induction of apoptosis by compound and TNFα combination. HepG2 human liver cancer cells grown in medium with 1% FBS were treated with TNFα (10 ng/mL) and/or indicated compound (40 μM) for 4 h and analyzed by immunoblotting. FIG. 10C depicts dose dependent effect of K-8008 on apoptosis induction. A549 lung cancer cells cultured in medium with 1% FBS were treated with TNFα (10 ng/mL) in the presence or absence of the indicated concentration of compound K-8008 for 4 h and analyzed by immunoblotting.

FIG. 11 depicts K-8008 inhibits TNFα induced tRXRα-p85a interaction. Co-immunoprecipitation assays were carried out in PC3 cells to determine tRXRα interaction with p85a. Cells treated with TNFα and/or K-8008 (40 μM) for 1 h were analyzed for tRXRα and p85a interaction by immunoprecipitation assay using AN 197 anti-RXRα antibody. The co-immunoprecipitates were then subjected to immunoblotting analysis for tRXRα expression and its co-precipitated p85a by AN 197 anti-RXRα and anti-p85α antibodies, respectively.

FIG. 12A depicts superposition of the RXRα LBD tetramer in complex with K-8008 and the apo protein structure (PDB 1G1U). FIG. 12B depicts superposition of the RXRα LBD tetramer in complex with K-8008 and the RXRα LBD tetramer in complex with K-8012.

FIG. 13A depicts distance between the binding region of K-8008 and the binding region of 9-cis-RA. Distance between the centroids of bound 9-cis-RA and the bound K-8008 is measured. FIG. 13B depicts distance between closest N of the tetrazol of K-8008 and the backbone N of residue of Phe438. FIG. 13C depicts locations of residues that contribute both to the K-8008 binding and the 9-cis-RA binding.

### DETAILED DESCRIPTION

Embodiments provided herein relate to methods and compositions for treating cancer. Some embodiments relate to certain compounds having activity against retinoid X receptor-alpha (RXRα). Some embodiments included designing or identifying a compound that binds to human RXRα protein, such as the ligand binding domain (LBD) of human RXRα protein.

Certain non-steroidal anti-inflammatory drugs (NSAIDs), including Etodolac and Sulindac, can bind to RXRα and modulate its biological activities. Interestingly, Sulindac but not 9-cis-RA can inhibit the binding of an N-terminally-truncated RXRα protein (tRXRα) to the p85α regulatory subunit of phosphatidylinositol-3-OH kinase (PI3K), leading to inhibition of tumor necrosis factor-a (TNFα)-activated PI3K/AKT pathway (Zhou et al., 2010). Certain compounds such as K-80003 include a new generation of RXRα-specific molecules for therapeutic application and mechanistic studies of RXRα (Wang et al., 2013; Zhou et al., 2010). These results identify Sulindac and other compounds as unique regulators of tRXRα activity through an undefined binding mechanism. Described herein are embodiments related to synthesis and characterization of compounds including K-8008 and K-8012, which exhibited improved activity in inhibiting tRXRα-mediated PI3K/AKT signaling pathway. Moreover, X-ray crystallographic studies of the LBD of RXRα in complex with K-8008 or K-8012 revealed that both compounds bound to the RXRα LBD in its tetrameric form via a novel site outside of the classical RXRα LBP, providing a new strategy for developing RXRα-based agents for cancer therapy.

### Compounds

Some embodiments of the methods and compositions provided herein include the following compounds. Some embodiments include a compound of Formula (I) having the structure:

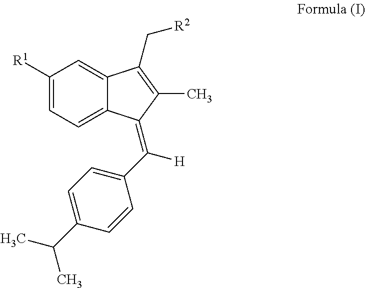

or tautomers thereof,

wherein:

R¹ is H or halogen; and

R² is C(═O)OH or

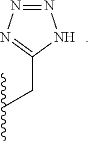 [SMILES: *Cc1nnn[nH]1]

In some embodiments, R¹ is H.

In some embodiments, R¹ is F.

In some embodiments, R² is C(═O)OH.

In some embodiments, R² is

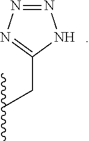 [SMILES: *Cc1nnn[nH]1]

In some embodiments, a compound of Formula (I) has the following structure:

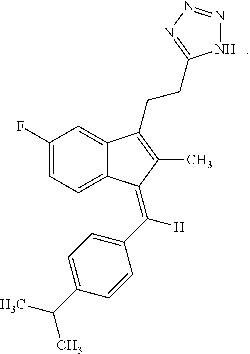 [SMILES: CC1=C(CCc2nnn[nH]2)c2cc(F)ccc2/C1=C\c1ccc(C(C)C)cc1]

In some embodiments, a compound of Formula (I) has the following structure:

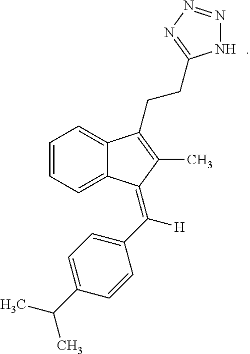 [SMILES: CC1=C(CCc2nnn[nH]2)c2ccccc2/C1=C\c1ccc(C(C)C)cc1]

The term “halogen atom” or “halogen” as used herein, means any one of the radio-stable atoms of column 7 of the Periodic Table of the Elements, such as, fluorine, chlorine, bromine and iodine.

In some embodiments, depending upon the substituents present, compounds can be in a form of a pharmaceutically acceptable salt. The terms “pharmaceutically acceptable salt” as used herein are broad terms, and is to be given its ordinary and customary meaning to a person of ordinary skill in the art (and is not to be limited to a special or customized meaning), and refers without limitation to salts prepared from pharmaceutically acceptable, non-toxic acids or bases. Suitable pharmaceutically acceptable salts include metallic salts, e.g., salts of aluminum, zinc, alkali metal salts such as lithium, sodium, and potassium salts, alkaline earth metal salts such as calcium and magnesium salts; organic salts, e.g., salts of lysine, N,N′-dibenzylethylenediamine, chloroprocaine, choline, diethanolamine, ethylenediamine, meglumine (N-methylglucamine), procaine, and tris; salts of free acids and bases; inorganic salts, e.g., sulfate, hydrochloride, and hydrobromide; and other salts which are currently in widespread pharmaceutical use and are listed in sources well known to those of skill in the art, such as, for example, The Merck Index. Any suitable constituent can be selected to make a salt of the therapeutic agents discussed herein, provided that it is non-toxic and does not substantially interfere with the desired activity.

In some embodiments, compounds can include isomers, racemates, optical isomers, enantiomers, diastereomers, tautomers, and cis/trans conformers. All such isomeric forms are included within preferred embodiments, including mixtures thereof. In some embodiments, compounds may have chiral centers, for example, they may contain asymmetric carbon atoms and may thus exist in the form of enantiomers or diastereoisomers and mixtures thereof, e.g., racemates. Asymmetric carbon atom(s) can be present in the (R)-, (S)-, or (R,S)-configuration, preferably in the (R)- or (S)-configuration, or can be present as mixtures. Isomeric mixtures can be separated, as desired, according to conventional methods to obtain pure isomers.

In some embodiments, compounds can be in amorphous form, or in crystalline forms. The crystalline forms of the compounds of preferred embodiments can exist as polymorphs, which are included in preferred embodiments. In addition, some of the compounds of preferred embodiments may also form solvates with water or other organic solvents. Such solvates are similarly included within the scope of the preferred embodiments.

In some embodiments, compounds described herein can be labeled isotopically. Substitution with isotopes such as deuterium may afford certain therapeutic advantages resulting from greater metabolic stability, such as, for example, increased in vivo half-life or reduced dosage requirements. Each chemical element as represented in a compound structure may include any isotope of said element. For example, in a compound structure a hydrogen atom may be explicitly disclosed or understood to be present in the compound. At any position of the compound that a hydrogen atom may be present, the hydrogen atom can be any isotope of hydrogen, including but not limited to hydrogen-1 (protium) and hydrogen-2 (deuterium). Thus, reference herein to a compound encompasses all potential isotopic forms unless the context clearly dictates otherwise.

### Certain Synthetic Methods

Some embodiments of the methods and compositions provided herein include synthesis of compounds such as K-8008 and K-8012. In some embodiments, synthetic methods can include the following scheme.

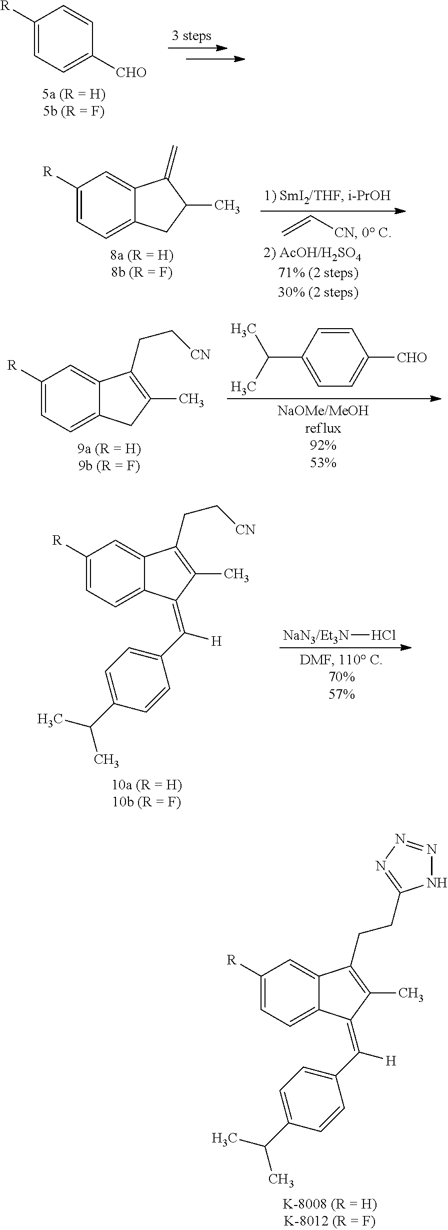

### 3-(2-Methyl-1H-inden-3-yl)propanenitrile (9a)

A solution of the 2-methylinden-1-one 8a (500.0 mg, 3.42 mmol), and iso-propanol (1.3 mL, 17.1 mmol), and acrylonitrile (2.26 mL, 34.2 mmol) in anhydrous THF (10.0 mL) was purged with argon for 20 min and cooled to 0° C. Then, A SmI₂ (10.3 mmol) solution in THF (103 mL) was added through transfer needle. After another 10 min, the reaction was quenched with a saturated aqueous NaHCO₃ (10 mL). The resulting mixture was extracted with Et₂O (20 mL×3). The combined organic layers were washed with a saturated aqueous Na₂S₂O₄, dried over anhydrous Na₂SO₄, filtered, and concentrated under reduced pressure. To the residue was added HOAc/H₂SO₄ (10/1, 5.0 mL). Then, after stirring overnight at room temperature, the mixture was extracted with EtOAc (15 mL×3). The combined extracts were washed successively with water, saturated NaHCCL, and brine, dried over Na₂SO₄, filtered, and concentrated under reduced pressure. The residue was purified by flash column chromatography on silica gel (ethyl acetate:PE=1:50) to afford compound 9a (443 mg, 71%) as a white solid. M.p. 53-54° C. (ethyl acetate/PE); IR (film): vₘₐₓ3430, 3015, 2909, 2244, 1631, 1607, 1467, 1394, 1028 cm⁻¹; ¹H NMR (400 MHz, CDCl₃) δ 2.14 (s, 3H, C═CCH₃), 2.57 (t, J=7.4 Hz, 2H, CH₂CH₂CN), 2.89 (t, J=7.4 Hz, 2H, CH₂CH₂CN), 3.33 (s, 2H, ArCH₂C═C), 7.12-7.18 (m, 2H, Ar—H), 7.24-7.29 (m, 1H, Ar—H), 7.37-7.42 (m, 1H, Ar—H) ppm; ¹³C NMR (100 MHz, CDCl₃) δ 14.1, 16.6, 21.4, 42.8, 117.4, 119.4, 123.6, 124.1, 126.2, 133.1, 141.8, 142.4, 145.0 ppm; MS (ESI) m/z 206.1 (M+Na⁺); HRMS (ESI) calcd for C₁₃H₁₃NNa⁺ [M+Na⁺]: 206.0940; found: 206.0943.

### 3-(5-Fluoro-2-methyl-1//-inden-3-yl)propanenitrile (9b)

A solution of compound 8b (300.0 mg, 1.8 mmol), and iso-propanol (0.7 mL, 9.0 mmol), and acrylonitrile (1.2 mL, 18.0 mmol) in THF (4 mL) was purged with argon for 20 min and cooled to 0° C. A SmI₂ (5.4 mmol) solution in THF (54 mL) was added through transfer needle. After 5 min, the reaction was quenched with saturated aqueous Na₂CO₃ (10 mL). The resulting mixture was extracted with Et₂O (15 mL×3). The combined organic phases were washed with brine, dried over anhydrous Na₂SO₄, filtered and concentrated under reduced pressure. To the residue was added HOAc/H₂SO₄ (10/1, 3.0 mL). After stirring for 4 h at room temperature, the mixture was extracted with EtOAc (15 mL×3). The combined extracts were washed successively with saturated NaHCO₃ and brine, dried over Na₂SO₄, filtered, and concentrated under reduced pressure. The residue was purified by flash column chromatography on silica gel (ethyl acetate:PE=1:50) to afford compound 9b as a white solid (108 mg, 30%). M.p. 91-92° C. (hexane/EtOAc); IR (film): vₘₐₓ 2915, 2247, 1610, 1592, 1476, 1275, 1190, 1165 cm⁻¹; ¹H NMR (400 MHz, CDCl3) δ 2.16 (s, 3H, C═CCH₃), 2.57 (t, J=7.3 Hz, 2H, CH₂CH₂CN), 2.86 (t, J=7.3 Hz, 2H, CH2CH2CN), 3.31 (s, 2H, CH₂C═C), 6.79-6.88 (m, 2H, Ar—H), 7.27-7.32 (m, 1H, Ar—H) ppm; ¹³C NMR (100 MHz, CDCl3) δ 14.3, 16.6, 21.3, 41.2, 104.8 (d, J_{C}-f=24.0 Hz), 110.5 (d, 7_{C}-f=23.0 Hz), 119.2, 124.17 (d, J_{C}-f=9.0 Hz), 132.8, 137.5, 146.9 (d, J_{C}-f=9.0 Hz), 162.4 (d, 7_{C}-f=241.0 Hz) ppm; MS (ESI) m/z 224.1 (M+Na⁺ 100%); HRMS (ESI) calcd for C₁₃H₁₂FNNa⁺[M+Na]⁺: 224.0846; found: 224.0848.

### (Z)-3-(1-(4-iso-Propylbenzylidene)-2-niethyl-1H-inden-3-yl)propanenitrile (10a)

To a solution of compound 9a (238 mg, 1.3 mmol) in MeOH (4.0 mL) was added 2.5 N NaOMe (1.6 mL, 4.0 mmol) at room temperature to get an orange mixture. After stirring for 30 min, to the mixture was added 4-isopropylbenzaldehyde (0.3 mL, 2.0 mmol). The resulting mixture was refluxed at 80° C. for 4 h. After concentration under reduced pressure, the residue was acidified with a IN HCl solution to pH 4.0˜6.0. After stirring for another 0.5 h at room temperature, the mixture was extracted with EtOAc (15 mL×3). The combined organic layers were dried over anhydrous Na₂SO₄, filtered, and concentrated under reduced pressure. The residue was purified by flash chromatography (ethyl acetate:PE=1:50) to afford indene derivative 10a as a yellow solid (374 mg, 92%). M.p. 88-90° C. (Et2O/hexane); IR (film): vₘₐₓ 3022, 2957, 2241, 1604, 1506, 1461, 1330, 1055 cm⁻¹; ¹H NMR (400 MHz, CDCl₃) δ 1.23 (d, J=6.9 Hz, 6H, CH(CH₃)₂), 2.14 (s, 3H, C═CCH₃), 2.51 (t, J=7.4 Hz, 3H, CH₂CH₂CN), 2.83-2.93 (m, 1H, CH(CH₃)₂), 2.88 (t, J=7.4 Hz, 2H, CH₂CH₂CN), 6.80-6.88 (m, 1H, Ar—H), 6.97-7.03 (m, 1H, Ar—H), 7.07-7.12 (m, 1H, Ar—H), 7.12 (s, 1H, vinyl-H), 7.18-7.23 (m, 2H, Ar—H), 7.36-7.43 (m, 3H, Ar—H) ppm; ¹³C NMR (100 MHz, CDCl₃) δ 10.4, 16.6, 21.7, 23.9 (2C), 34.0, 117.0, 119.3, 123.0, 124.6, 126.5, 129.4, 131.1, 134.0, 134.4, 134.7, 136.0, 140.6, 143.1, 149.1 ppm; MS (ESI) m/z 336.2 (M+Na⁺); HRMS (ESI) calcd for C₂₃H₂₃NNa⁺[M+Na⁺]: 336.1723; found: 336.1729.

### (Z)-3-(5-Fluoro-1-(4-isopropylbenzylidene)-2-methyl-1H-inden-3-yl)propanenitrile (10b)

To a solution of compound 9b (261 mg, 1.3 mmol) in MeOH (4.0 mL) was added 2.5 N NaOMe (1.6 mL, 4.0 mmol) at room temperature to get an orange mixture. After stirring for 30 min, to the mixture was added 4-isopropylbenzaldehyde (0.3 mL, 2.0 mmol). The resulting mixture was refluxed at 80° C. for 4 h. After concentrated under reduced pressure, the residue was acidified with a IN HCl solution to pH 4.0-6.0. After stirring for another 0.5 h at room temperature, the mixture was extracted with EtOAc (15 mL×3). The combined organic layers were dried over anhydrous Na₂SO₄, filtered, and concentrated under reduced pressure. The residue was purified by flash chromatography (ethyl acetate:PE=1:50) to afford indene derivative 10b as a yellow solid (228 mg, 53%). M.p. 108-109° C. (hexane/EtOAc); IR (film): vmax 2957, 2927, 2866, 2247, 1598, 1464, 1199, 1162, 1138, 1055, 1016 cm⁻¹; ¹H NMR (400 MHz, CDCl3) δ 1.32 (d, 7=6.9 Hz, 6H, CH(CH₃)₂), 2.24 (s, 3H, C═CCH₃), 2.60 (t, 7=7.4 Hz, 2H, CH₂CH_CN), 2.93 (t, 7=7.4 Hz, 2H, CH₂CH₂CN), 2.98 (sept, 7=6.9 Hz, 1H, CH (CH₃)₂), 6.58-6.65 (m, 1H, Ar—H), 6.75-6.80 (m, 1H, Ar—H), 7.21 (s, 1H, vinyl-H), 7.28-7.33 (m, 2H, Ar—H), 7.39-7.48 (m, 3H, Ar—H) ppm; ¹³C NMR (100 MHz, CDCl₃) δ 10.5, 16.6, 21.6, 23.9, 34.0, 104.7 (d, 7_{C}.f=23.0 Hz), 110.6 (d, 7_{C}-f=22.0 Hz), 119.1, 124.0 (d, 7_{C-F}=8.0 Hz), 126.5 (2C), 129.4 (2C), 130.2, 131.1, 133.68, 133.78, 138.2, 139.6, 145.47 (d, 7_{C-F}=8.0 Hz), 149.3, 163.0 (d, J_{c},f=244.0 Hz) ppm; MS (ESI) m/z 354.2 (M+Na⁻, 100%); HRMS (ESI) calcd for C₂₃H₂₂FNNa⁺ [M+Na⁺]: 354.1628; found: 354.1625.

### (Z)-5-(2-(1-(4-iso-Propylbenzylidene)-2-methyl-1H-inden-3-yl)ethyl)-1H-tetrazole (K-8008)

A flask (10 mL) was charged with nitrile 10a (45 mg, 0.14 mmol) and dry DMF (0.8 mL), triethylamine hydrochloride (110 mg, 0.80 mmol) and sodium azide (52 mg, 0.80 mmol) were added to the solution under nitrogen. The mixture was heated for 40 h at 110° C., then cooled to the room temperature, concentrated in vacuo and diluted with water (10 mL). The aqueous solution was then acidified to pH 2.0 using concentrated HCl, extracted with EtOAc. The combined organic layers were washed with brine, dried over anhydrous Na₂SO₄, filtered, and concentrated under reduced pressure. The residue was purified by flash chromatography (ethyl acetate:PE=1:1) to afford crude K-8008 (36 mg, 70%). M.p. 173-175° C. (CH₂C₂/hexane); IR (film): vₘₐₓ 3136, 3022, 2957, 2737, 2616, 1911, 1598, 1564, 1463, 1326, 1254 1049 cm⁻¹; ¹H NMR (400 MHz, CDCl₃) δ 1.29 (d, 7=6.9 Hz, 6H, CH(CH₃)₂), 1.92 (s, 3H, C═CCH₃), 2.90-2.99 (m, 1H, CH(CH₃)₂), 3.05 (t, 7=7.1 Hz, 2H, CH₂CH₂-Tetrazole), 3.27 (t, 7=7.1 Hz, 2H, CH₂CH₂-Tetrazole), 6.85-6.92 (m, 1H, Ar—H), 7.05-7.14 (m, 3H, vinyl-H, Ar—H), 7.20-7.28 (m, 2H, Ar—H), 7.35-7.49 (m, 3H, Ar—H) ppm; ¹³ C NMR (100 MHz, CDCl₃) δ 9.9, 22.6, 23.9 (2C), 29.7, 34.0, 117.4, 122.9, 124.6, 126.5, 127.8, 129.4, 130.9, 133.9, 134.4, 135.6, 135.7, 140.5, 143.4, 149.1, 155.9 ppm; MS (ESI) m/z 379.2 (M+Na⁺; HRMS (ESI) calcd for C₂₃H₂₄N₄Na⁺ [M+Na⁺] 379.1893; found 379.1894.

### (Z)-5-(2-(5-Fluoro-1-(4-isopropylbenzylidene)-2-methyl-1H-inden-3-yl)ethyl)-1H-tetrazole (K-8012)

A flask (10 mL) was charged with the nitrile 10b (96 mg, 0.29 mmol) and dry DMF (3.0 mL), triethylamine hydrochloride (200 mg, 1.45 mmol) and sodium azide (94.3 mg, 1.45 mmol) were added to the solution under nitrogen. The mixture was heated for 40 h at 110° C., then cooled to the room temperature, concentrated in vacuo and diluted with water (10 mL). The aqueous solution was then acidified to pH 2.0 using concentrated HCl, extracted with EtOAc. The combined organic layers were washed with brine, dried over anhydrous Na₂SO₄, filtered, and concentrated under reduced pressure. The residue was purified by flash chromatography to afford compound K-8012 as a yellow solid (62 mg, 57%). M.p. 201-202° C. (hexane/EtOAc); IR (film): vₘₐₓ 3143, 2964, 2930, 2866, 2731, 2619, 2454, 1597, 1464, 1266, 1180, 1134, 1101, 1052 cm⁻¹; ¹H NMR (400 MHz, Methanol-r/₄) δ 1.29 (d, J=6.8 Hz, 6H, CH(CH3)₂), 1.96 (s, 3H, C═CCH₃), 2.96 (sept, J−6.8 Hz, 1H, CH(CH₃)₂), 3.03 (t, J=7.1 Hz, 2H, CH₂CH₂-Tetrazole), 3.19 (t, J=7.1 Hz, 2H, CH₂CH₂-Tetrazole), 6.50-6.60 (m, 1H, Ar—H), 6.86-6.94 (m, 1H, Ar—H), 7.17 (s, 1H, vinyl-H), 7.24-7.35 (m, 3H, Ar—H), 7.37-7.44 (m 2H, Ar—H) ppm; ¹³C NMR (100 MHz, Methanol-d₄) δ 10.0, 23.5, 24.3, 24.8, 35.3, 106.0 (d,/_{C}-f=24.0 Hz), 111.1 (d,_{/c}_{^{-F}}=23.0 Hz), 124.8 (d, 7_{C}-f=9.0 Hz), 127.6 (2C), 130.5 (2C), 131.5, 131.6, 135.4, 136.67 (d, J_{C}-f=2.0 Hz), 138.5, 141.2, 147.69 (d, 7_{C}-f=9.0 Hz), 150.5, 157.3, 164.5 (d, 7_{C}-f=243.0 Hz) ppm; MS (ESI) m/z 397.2 (M+Na⁺, 100%); HRMS (ESI) calcd for C₂₃H₂₃FN₄Na⁺[M+Na⁺]: 397.1799; found: 397.1804.

### Pharmaceutical Compositions

Some embodiments of the methods and compositions provided herein include pharmaceutical compositions comprising the compounds provided herein. In some embodiments, pharmaceutical compositions can be in admixture with a suitable carrier, diluent, or excipient such as sterile water, physiological saline, glucose, or the like, and can contain auxiliary substances such as wetting or emulsifying agents, pH buffering agents, gelling or viscosity enhancing additives, preservatives, flavoring agents, colors, and the like, depending upon the route of administration and the preparation desired. See, e.g., “Remington: The Science and Practice of Pharmacy”, Lippincott Williams & Wilkins; 20th edition (Jun. 1, 2003) and “Remington's Pharmaceutical Sciences,” Mack Pub. Co.; 18^{th} and 19^{th} editions (December 1985, and June 1990, respectively). Such preparations can include complexing agents, metal ions, polymeric compounds such as polyacetic acid, polyglycolic acid, hydrogels, dextran, and the like, liposomes, microemulsions, micelles, unilamellar or multilamellar vesicles, erythrocyte ghosts or spheroblasts. Suitable lipids for liposomal formulation include, without limitation, monoglycerides, diglycerides, sulfatides, lysolecithin, phospholipids, saponin, bile acids, and the like. The presence of such additional components can influence the physical state, solubility, stability, rate of in vivo release, and rate of in vivo clearance, and are thus chosen according to the intended application, such that the characteristics of the carrier are tailored to the selected route of administration.

In some embodiments, pharmaceutical compositions can be isotonic with the blood or other body fluid of the recipient. The isotonicity of the compositions can be attained using sodium tartrate, propylene glycol or other inorganic or organic solutes. Sodium chloride is particularly preferred. Buffering agents can be employed, such as acetic acid and salts, citric acid and salts, boric acid and salts, and phosphoric acid and salts. Parenteral vehicles include sodium chloride solution, Ringer's dextrose, dextrose and sodium chloride, lactated Ringer's or fixed oils. Intravenous vehicles include fluid and nutrient replenishers, electrolyte replenishers (such as those based on Ringer's dextrose), and the like.

In some embodiments, viscosity of the pharmaceutical compositions can be maintained at the selected level using a pharmaceutically acceptable thickening agent. Methylcellulose is preferred because it is readily and economically available and is easy to work with. Other suitable thickening agents include, for example, xanthan gum, carboxymethyl cellulose, hydroxypropyl cellulose, carbomer, and the like. The preferred concentration of the thickener will depend upon the thickening agent selected. An amount is preferably used that will achieve the selected viscosity. Viscous compositions are normally prepared from solutions by the addition of such thickening agents.

In some embodiments, a pharmaceutically acceptable preservative can be employed to increase the shelf life of the pharmaceutical compositions. Benzyl alcohol can be suitable, although a variety of preservatives including, for example, parabens, thimerosal, chlorobutanol, or benzalkonium chloride can also be employed. A suitable concentration of the preservative is typically from about 0.02% to about 2% based on the total weight of the composition, although larger or smaller amounts can be desirable depending upon the agent selected. Reducing agents, as described above, can be advantageously used to maintain good shelf life of the formulation.

In some embodiments, pharmaceutical compositions can be administered in an intravenous or subcutaneous unit dosage form; however, other routes of administration are also contemplated. Contemplated routes of administration include but are not limited to oral, parenteral, intravenous, and subcutaneous. The inhibitors of preferred embodiments can be formulated into liquid preparations for, e.g., oral administration. Suitable forms include suspensions, syrups, elixirs, and the like. Particularly preferred unit dosage forms for oral administration include tablets and capsules. Unit dosage forms configured for administration once a day are particularly preferred; however, in certain embodiments it can be desirable to configure the unit dosage form for administration twice a day, or more.

### Methods for Inhibiting Tumor Cells

Some embodiments of the methods and compositions provided herein include methods of inhibiting a tumor cell. Some of the foregoing embodiments include contacting a tumor cell with a compound provided herein, such as K-8008 or K-8012. Some embodiments also include contacting the cell with an additional agent. In some embodiments, the additional agent comprises Tumor necrosis factor (TNFα). In some embodiments, the tumor cell can include a lung tumor cell, a prostate tumor cell, a breast tumor cell, a colon tumor cell, a colon tumor cell, a liver tumor cell, and a lung tumor cell. In some embodiments, the tumor cell is from a subject. In some embodiments, the tumor cell is in vivo. In some embodiments, the tumor cell is in vitro. In some embodiments, the tumor cell includes a cell of a cell line such as, PC3, ZR-75-1, HeLa, HCT-116, A549, MB231, HepG2, and CV-1. In some embodiments, the tumor cell is the tumor cell is mammalian. In some embodiments, the tumor cell is the tumor cell is human.

Some embodiments include treating and/or ameliorating a tumor in a subject. Some embodiments of the methods and compositions provided herein include methods of inhibiting a tumor. Some embodiments include reducing the growth of a tumor. Some embodiments include reducing the volume of a tumor. Some embodiments include reducing the number of tumor cells in a tumor. Some embodiments include comprising administering to a subject in need thereof an effective amount of the pharmaceutical composition comprising a compound provided herein. In some embodiments, the compound is K-80003, K-8008 or K-8012. Some embodiments also include contacting the cell with an additional agent. In some embodiments, the additional agent comprises Tumor necrosis factor (TNFα). In some embodiments, the cancer is liver cancer. In some embodiments, the subject is mammalian. In some embodiments, the subject is human.

### Method for Designing Compounds

Some embodiments of the methods and compositions provided herein include methods for designing a compound that binds to human RXRα protein. Some embodiments include accessing data comprising the structure of at least the ligand binding domain (LBD) of human RXRα protein; and modeling the binding of the compound to human RXRα protein using said data. Some embodiments also include predicting the likelihood that the compound binds to a hydrophobic region of the LBD that does not overlap with the binding site of 9-cis-retinoic acid. Some embodiments also include predicting that the compound does not change the conformation of the LBP. In some embodiments, the compound is predicted to bind to a region of the LBD comprising at least one residue selected from the group consisting of Ala271, Ala272, Trp305, Leu309, Leu326, Leu330, Leu433, Leu436, Phe437, Phe438, Ile442, Gly443, and Leu436. In some embodiments, the compound is designed de novo. In some embodiments, the compound is designed from a known chemical entity of fragment thereof. In some embodiments, the chemical entity is a compound provided herein. In some embodiments, the chemical entity is K-8008 or K-8012.

Some embodiments of methods for designing a compound that binds to human RXRα protein designing compounds using techniques of structure-based drug design. Structure-based drug design involves the rational design of ligand molecules to interact with the three-dimensional (3-D) structure of target receptors; the ultimate goal being to identify or design molecules with 3-D complementarity to the target protein, such as human RXRα protein, such as the LBD of human RXRα protein (Kirkpatrick et al. (1999) Comb. Chem. High Throughput Screen. 2: 211-21). In some embodiments, computational procedures may be used to suggest ligands that will bind to human RXRα protein, such as the LBD of human RXRα protein. In some embodiments, a compound can be designed de novo. In more embodiments, a compound can be designed using the structure of a compound known to interact with human RXRα protein, such as the LBD of human RXRα protein. Interactive graphics approaches explore new ligand designs manually in ways that might involve, for example, modification of groups on the ligand to optimize complementarity with receptor/enzyme subsites, optimization of a transition state to reflect data from mechanistic studies, replacement of peptide bonds with groups that improve hydrolytic stability while maintaining key hydrogen bond interactions, or linking of adjacent side groups to increase the rigidity of the ligand (Whittle and Blundell (1994) Annu. Rev. Biophys. Biomol. Struct. 23: 349-75). Most of these steps can now be done using systematic computational approaches that fall into three classes: 1) automated docking of whole molecules into receptor sites; 2) precalculating potentials at grid points and fitting molecules to these potentials; and 3) docking fragments and either joining them or growing them into real molecules (Whittle and Blundell (1994) Annu. Rev. Biophys. Biomol. Struct. 23: 349-75).

Numerous computer programs are available and suitable for rational drug design and the processes of computer modeling, model building, and computationally identifying, selecting and evaluating potential inhibitors in the methods described herein. These include, for example, SYBYL (available from TRIPOS, St. Louis Mo.), DOCK (available from University of California, San Francisco), GRID (available form Oxford University, UK), MCSS (available from Molecular Simulations Inc., Burlington, Mass.), AUTODOCK (available from Oxford Molecular Group), FLEX X (available from TRIPOS, St. Louis Mo.), CAVEAT (available from University of California, Berkeley), HOOK (available from Molecular Simulations Inc., Burlington, Mass.), and 3-D database systems such as MACCS-3D (available from MDL Information Systems, San Leandro, Calif.), UNITY (available from TRIPOS, St. Louis Mo.), and CATALYST (available from Molecular Simulations Inc., Burlington, Mass.).

Potential interactive compounds may also be computationally designed de novo using such software packages as LUDI (available from Biosym TechMA), and LEAPFROG (TRIPOS Associates, St. Louis, Mo.). Compound defammation energy and electrostatic repulsion, may be evaluated using programs such as GAUSSIAN 92, AMBER, QUANTA/CHARMM, and INSIGHT II/DISCOVER. These computer evaluation and modeling techniques may be performed on any suitable hardware including for example, workstations available from Silicon Graphics, Sun Microsystems, and the like. These techniques, methods, hardware and software packages are representative and are not intended to be comprehensive listing.

In some embodiments, other modeling techniques known in the art may also be employed. See for example, N. C. Cohen, Molecular Modeling in Drug Design, Academic Press (1996); Whittle and Blundell (1994) Annu. Rev. Biophys. Biomol. Struct. 23: 349-75; Grootenhuis et al. (1992) Bull. Soc. Chim. Belg. 101: 661; Lawrence and Davis (1992) Proteins Struct. Funct. Genet. 12: 31; Miranker and Karplus (1991) Proteins Struct. Funct. Genet. 11: 29). Other methods and programs include CLIX (a suite of computer programs that searches the Cambridge Data base for small molecules that have both geometrical and chemical complementarity to a defined binding site on a protein of known three-dimensional structure), and software identified at internet sites including the CAOS/CAMM Center Cheminformatics Suite and the NIH Molecular Modeling Home Page.

### Kits

Some embodiments of the methods and compositions provided herein include kits. Some embodiments include a compound provided herein and a pharmaceutically acceptable excipient. Some embodiments also include an additional therapeutic agent. In some embodiments, the additional therapeutic agent comprises Tumor necrosis factor (TNFα).

### EXAMPLES

### Example 1

### K-8008 and K-8012 are New Antagonists of RXRα

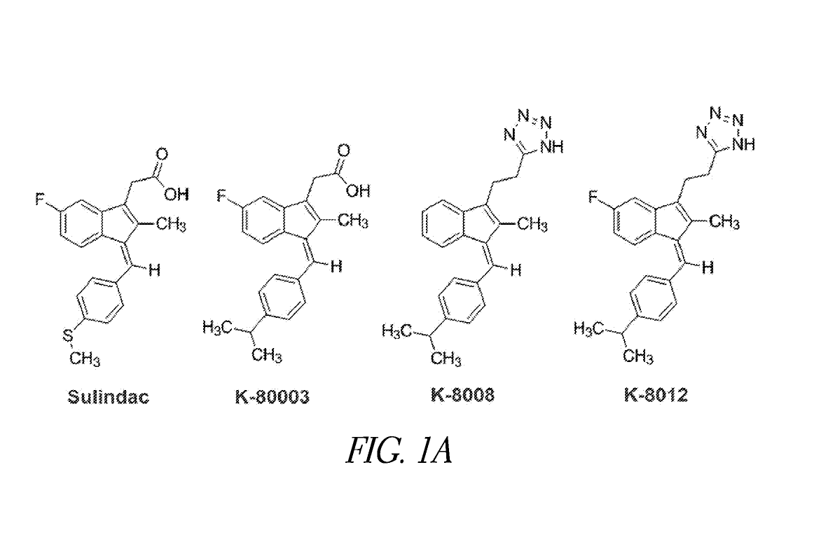

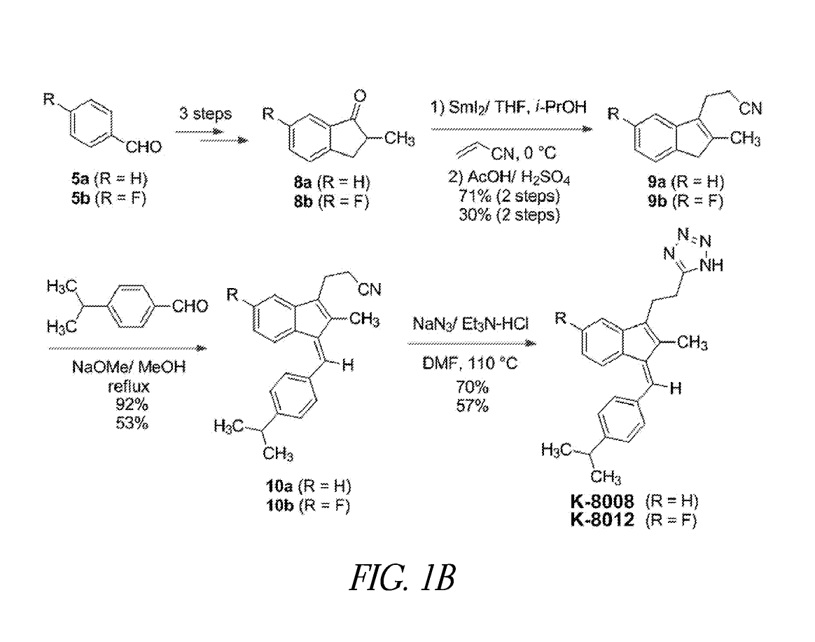

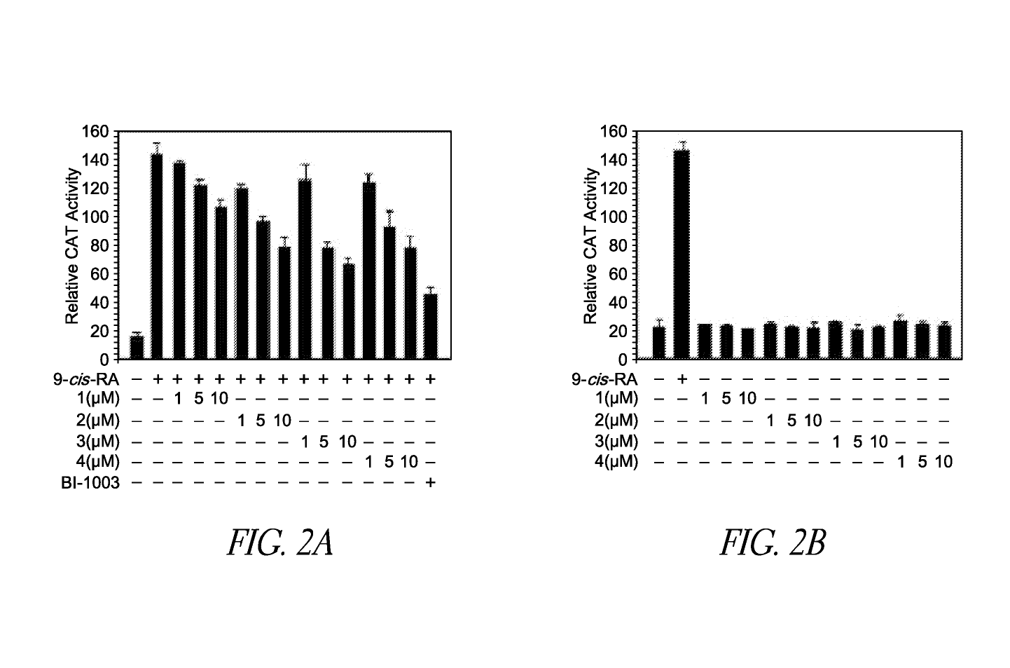

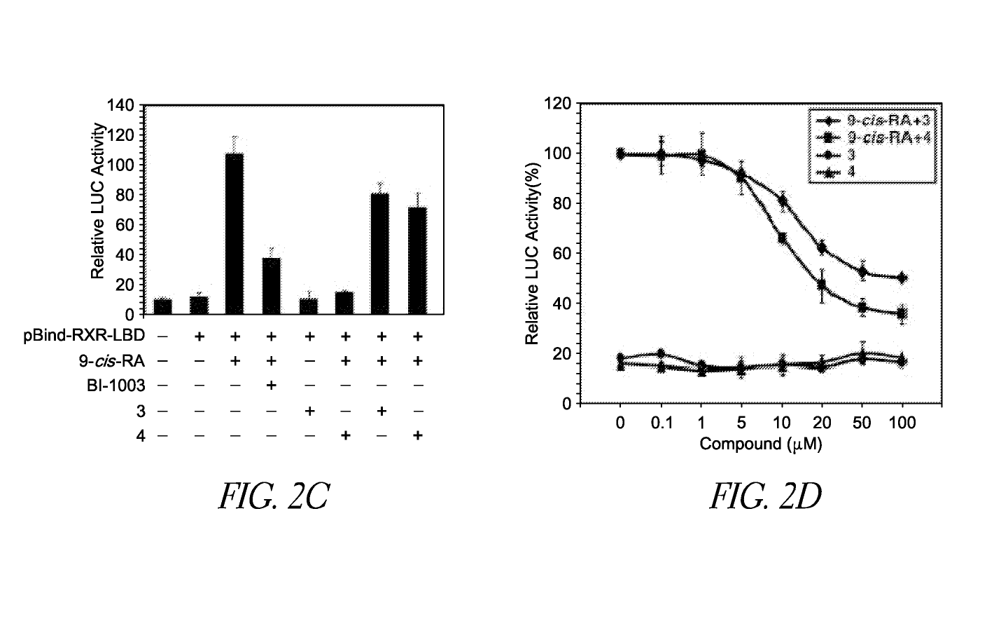

Compounds shown in FIG. 1A were initially evaluated by a reporter assay using a CAT reporter containing TREpal that known to bind to RXRα homodimer (Zhang et al., 1992). 9-cis-RA strongly induced the TREpal reporter activity, which was inhibited by BI-1003, a known RXRα antagonist (Lu et al., 2009). The compounds, K-8008 and K-8012, also exhibited inhibitory effect on 9-cis-RA-induced TREpal reporter activity in a concentration dependent manner (FIG. 2A), while they did not show any agonist activity at the concentrations used (FIG. 2B). The antagonist effect of K-8008 and K-8012 was much better than Sulindac (FIG. 2A), the Ga14-RXRα-LBD chimera and Ga14 reporter system was used to evaluate the inhibitory effect of K-8008 and K-8012 on 9-cis-RA-induced reporter activity. Cotransfection of Ga14-RXRα-LBD strongly activated the Ga14 reporter in the presence of 9-cis-RA, which was inhibited by BI-1003 as well as K-8008 and K-8012 (FIG. 2C). Dose response experiments showed that the IC50 values for K-8008 and K-8012 to inhibit 9-cis-RA-induced Ga14-RXRα-LBD transactivation were about 13.2 μM and 9.2 μM, respectively (FIG. 2D). Thus, K-8008 and K-8012 are new antagonists of RXRα.

### Example 2

### K-8008 and K-8012 Induce Apoptosis and Inhibit AKT Activation by Preventing tRXRα from Binding to p85α

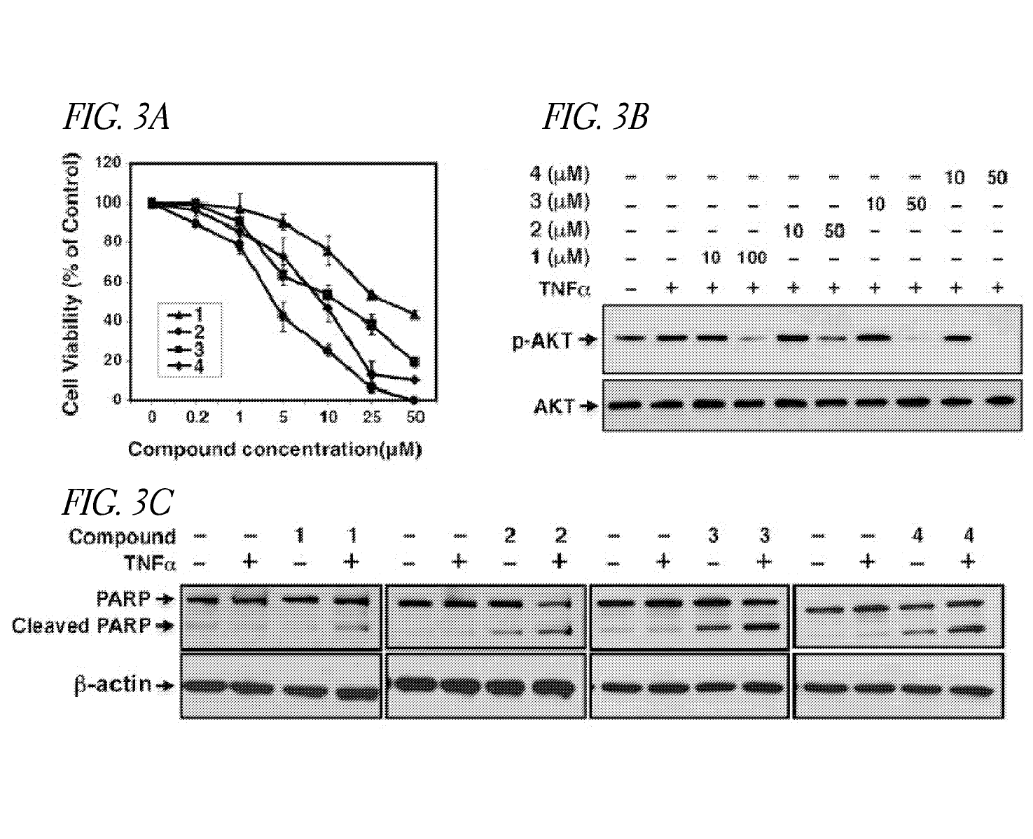

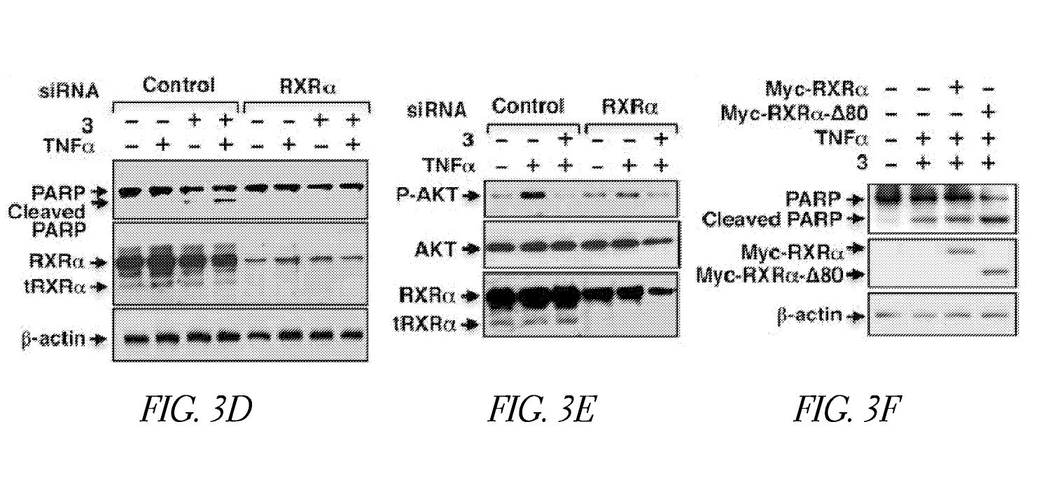

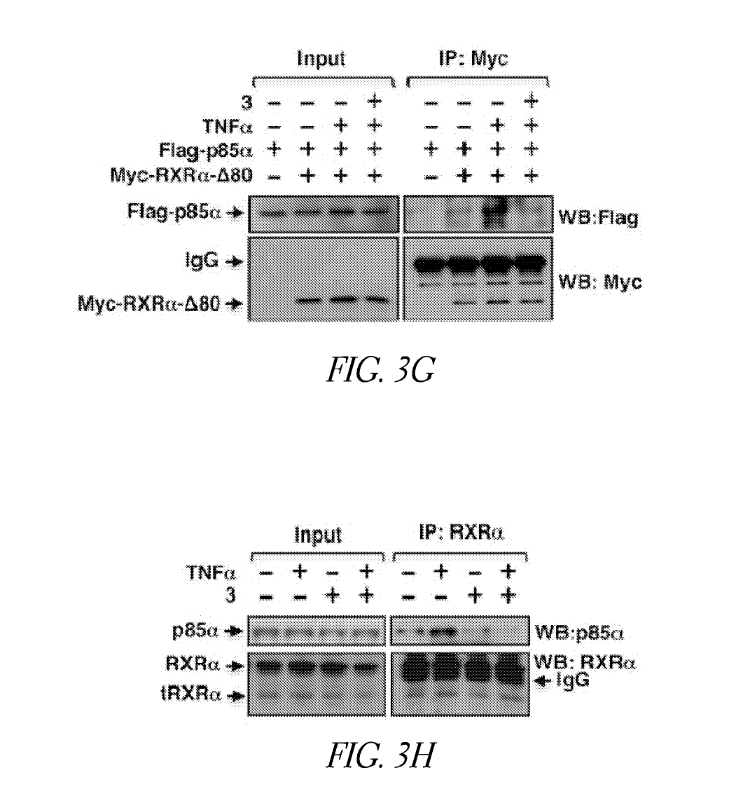

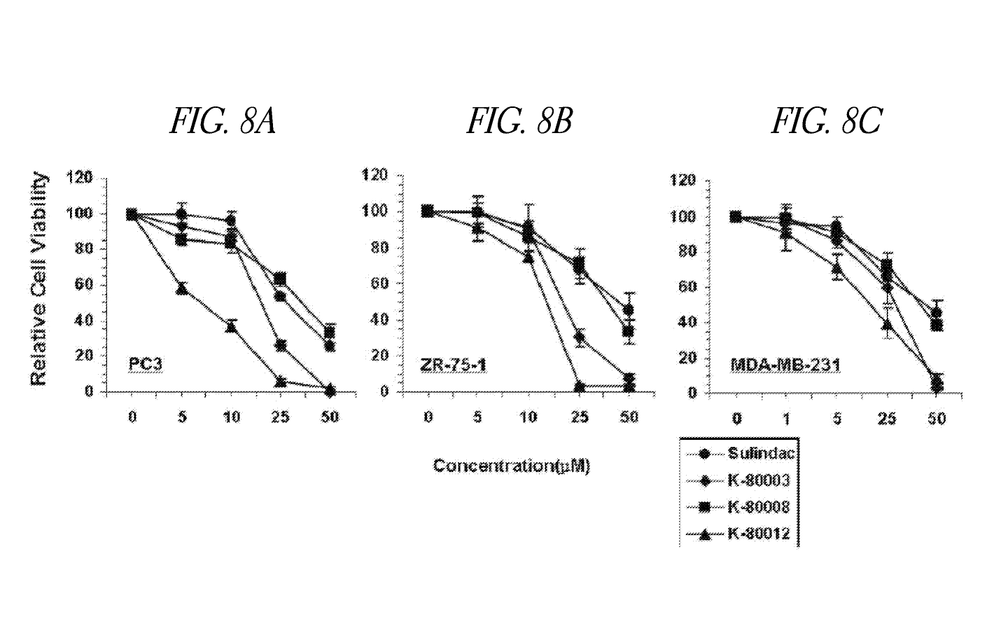

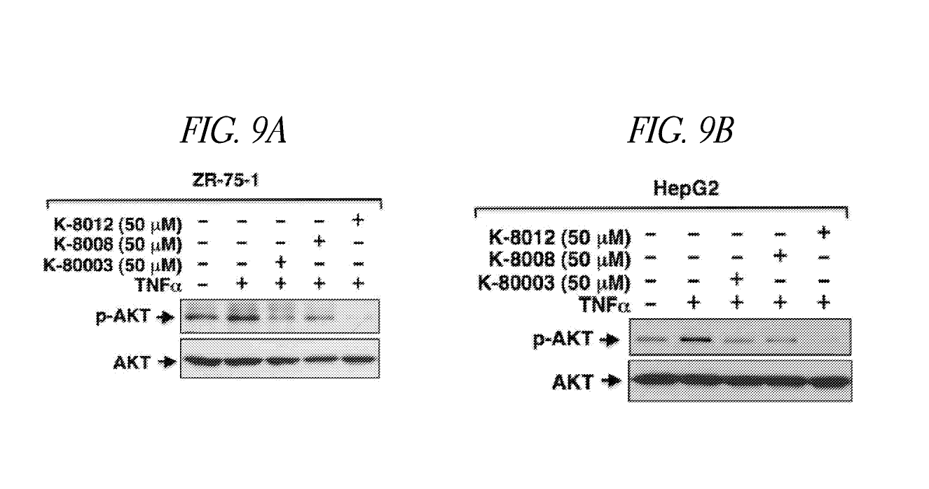

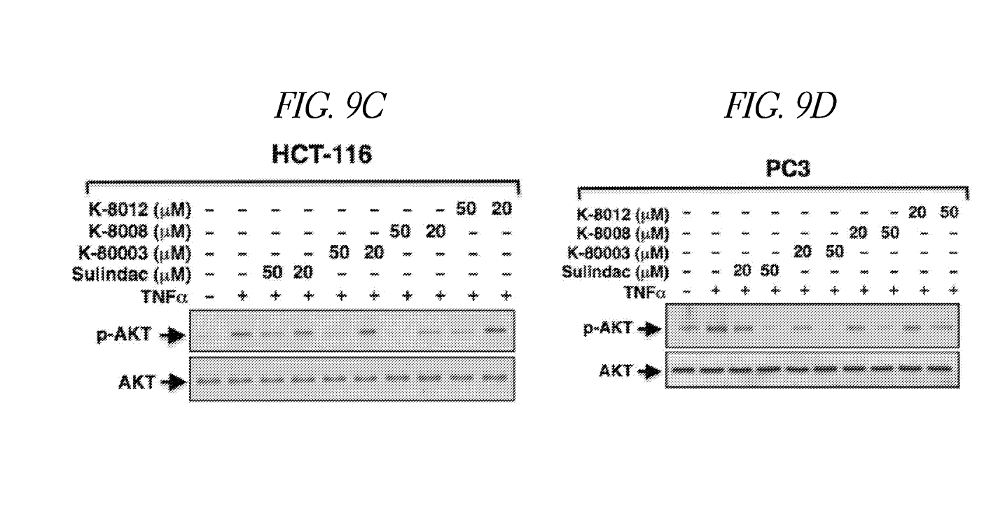

K-8008 and K-8012 were evaluated for their effect on the growth of cancer cells. Compared to Sulindac, K-8008 and K-8012 were much more effective in inhibiting the growth of various cancer cells, including A549 lung cancer (FIG. 3A), PC3 prostate cancer. ZR-75-1 and MB231 breast cancer cells (FIG. 8). A unique property of Sulindac and other compounds are their ability to inhibit TNFα-induced AKT activation (Zhou et al, 2010). Thus, A549 lung cancer cells were treated with TNFα in the absence or presence of K-8008 or K-8012. Treatment of cells with TNFα enhanced AKT activation as revealed by Western blotting (FIG. 3B). However, when cells were cotreated with either K-8008 or K-8012, the TNFα-induced AKT activation was suppressed in a dose dependent manner (FIG. 3B). Similar results were obtained in other cancer cell lines (FIG. 9).

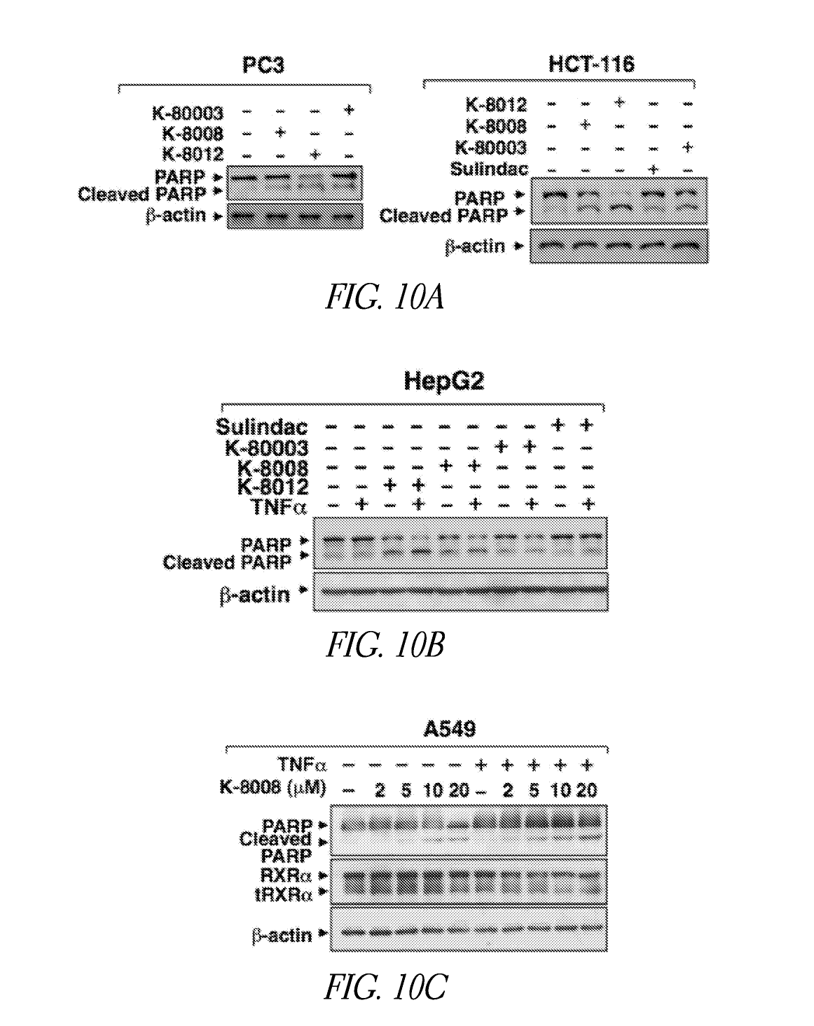

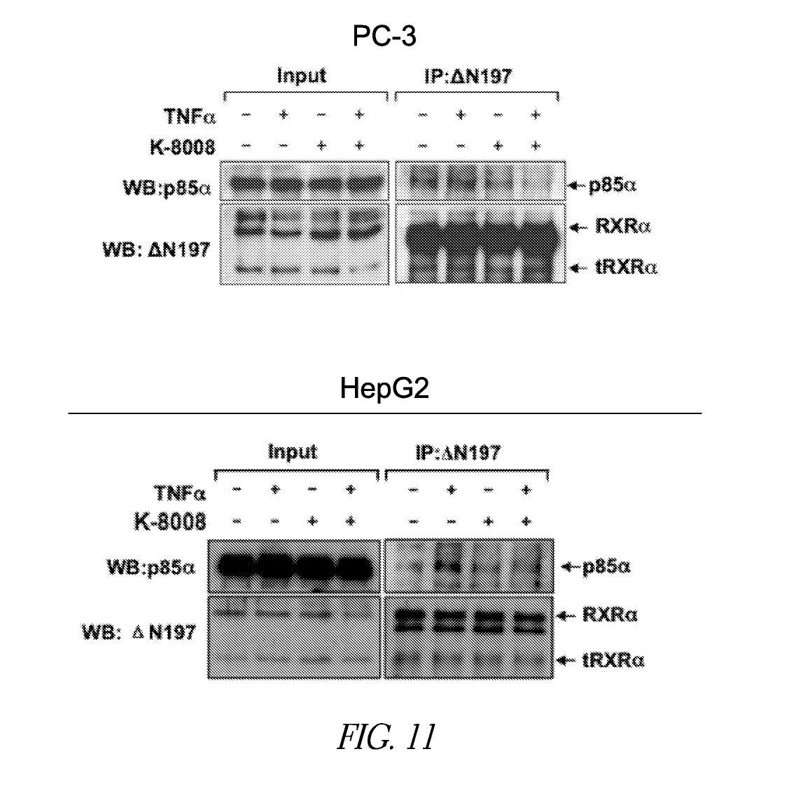

TNFα is a multifunctional cytokine that controls diverse cellular events such as cell survival and death (Balkwill, 2009; Wang and Lin, 2008). Inhibition of TNFα-induced AKT activation by Sulindac and other compounds in cancer cells led to a shift of TNFα signaling from survival to death (Zhou et ah, 2010). The effect of K-8008 and K-8012 alone or in combination with TNFα was examined on the cleavage of PARP, an indication of apoptosis in cancer cells (Lazebnik et ah, 1994). Treatment of A549 cells with TNFα did not have effect on PARP cleavage, whereas treatment with Sulindac or other compounds slightly induced PARP cleavage. Combination of Sulindac or other compounds with TNFα, however, caused a significant induction of PARP cleavage (FIG. 3C and FIG. 10). Thus, K-8008 and K-8012 could convert TNFα signaling from survival to death in cancer cells.

The growth inhibitory effect of K-8008 and K-8012 and the induction of apoptosis by K-8008 occurred at low micromolar concentrations, suggesting that they might exert their anti-cancer effects through RXRα binding. To address the issues, cancer cells were transfected with RXRα siRNA and evaluated for the effect of K-8008 on inducing PARP cleavage and inhibiting AKT activation. Knocking down RXRα expression by RXRα siRNA transfection significantly diminished the effect of K-8008 on inducing PARP cleavage (FIG. 3D) and inhibiting TNFα-induced AKT activation (FIG. 3E). To address the role of tRXRα, RXRα-A80, a RXRα mutant lacking its N-terminal 80 amino acids and mimicking tRXRα (Wang et al., 2013; Zhou et al., 2010), was transfected into HeLa cells. Transfection of RXRα-A80 but not the full-length RXRα enhanced the effect of K-8008 on inducing PARP cleavage in the presence of TNFα (FIG. 3F). Together, these results demonstrate that tRXRα plays a role in mediating the biological effects of K-8008.

Whether K-8008 could affect tRXRα interaction with p85α, an interaction known to activate AKT was examined (Zhou et al., 2010). HeLa cells were transfected with Myc-tagged RXRα-A80 and Flag-tagged p85α expression vectors and treated with or without TNFα and/or K-8008. Co-immunoprecipitation assays using anti-Myc antibody showed that Flag-p85α was co-immunoprecipitated together with Myc-RXRα-A80 in cells treated with TNFα (FIG. 3G). However, when cells were cotreated with K-8008, TNFα-induced interaction of Myc-RXRα-A80 with Flag-p85α was almost completely inhibited.

The effect of K-8008 on interaction of endogenous tRXRα with p85α in A549 cells was examined. Cell lysates prepared from A549 cells treated with TNFα in the presence or absence of K-8008 were analyzed by co-immunoprecipitation using A197 anti-RXRα antibody that recognizes both tRXRα and RXRα (Zhou et al., 2010). FIG. 3H showed that treatment of cells with TNFα promoted the interaction of endogenous tRXRα with p85α, consistent with previous finding (Zhou et al., 2010). When cells were co-treated with K-8008, the interaction was largely inhibited. Such an effect of K-8008 on inhibiting TNFα-induced p85α interaction with tRXRα was also observed in other cancer cell lines, including PC3 and HepG2 cells (FIG. 8). Together, these results demonstrate that K-8008 can induce TNFα-dependent apoptosis by suppressing the tRXRα-mediated activation of AKT through its inhibition of tRXRα interaction with p85α.

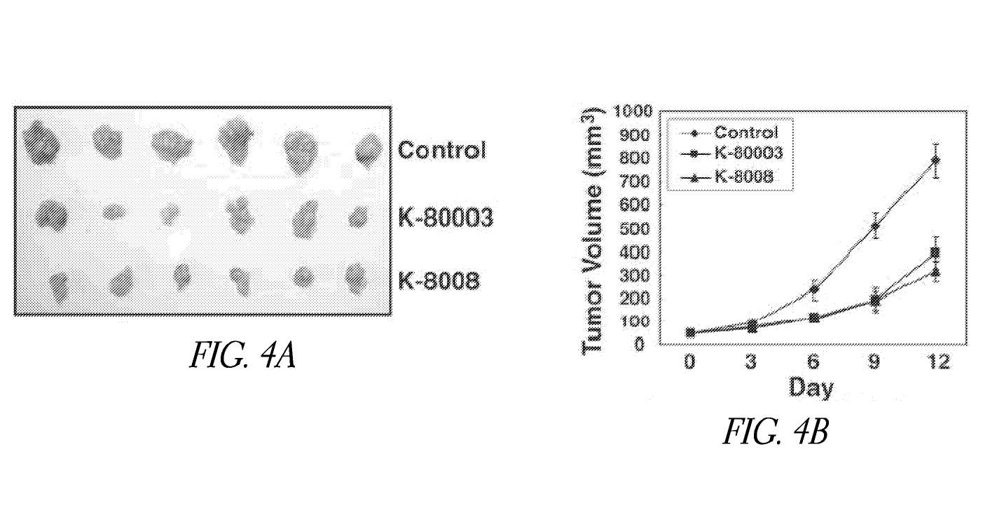

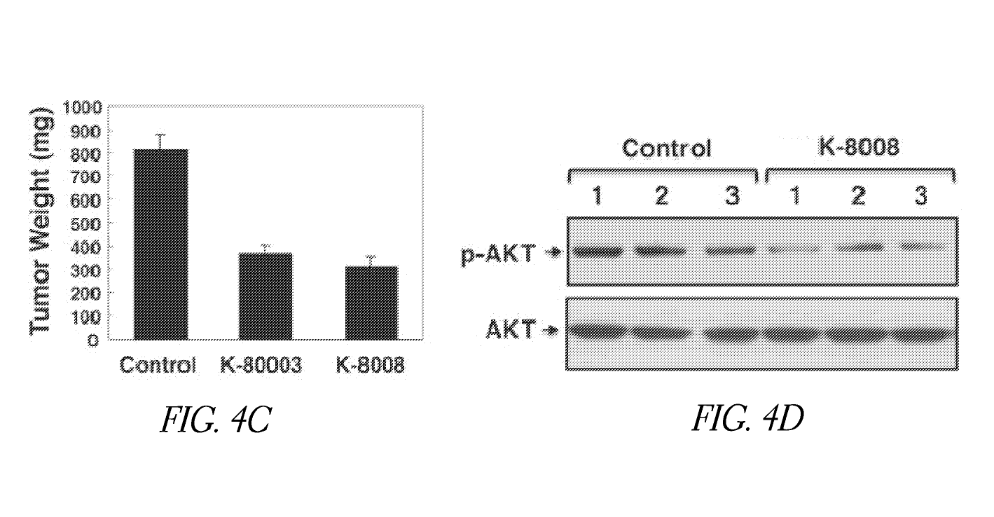

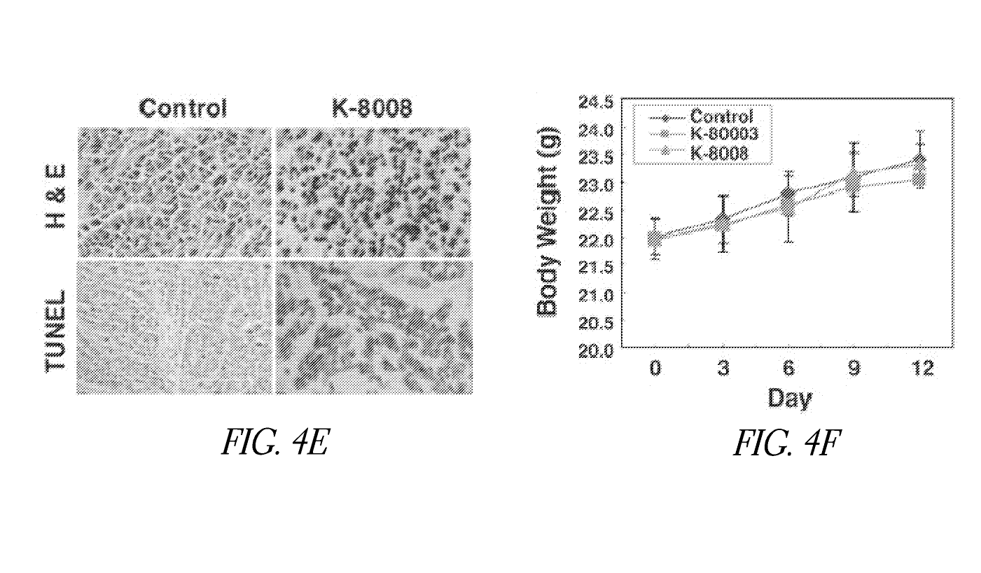

To further evaluate the anti-cancer effect of K-8008, mice with HepG2 tumor xenografts were treated with 20 mg/kg K-8008 or K-80003. Administration of K-8008 inhibited the growth of HepG2 tumor in a time dependent manner (FIG. 4A), resulting in a 61.23% reduction of tumor weight after a 12-day treatment (FIGS. 4B-4C), which was comparable with the inhibitory effect of K-80003 (54.84% reduction). Consistent with in vitro observations, examination of three tumors treated with or without K-8008 showed reduction of AKT activation by K-8008 (FIG. 4D). Moreover TUNEL staining revealed induction of apoptosis by K-8008 (FIG. 4E). Significantly, administration of either K-80003 or K-8008 did not show any apparent toxic effects such as loss of body weight (FIG. 4F).

### Example 3

### K-8008 and K-8012 do not Bind to the Classical LBP of RXRα

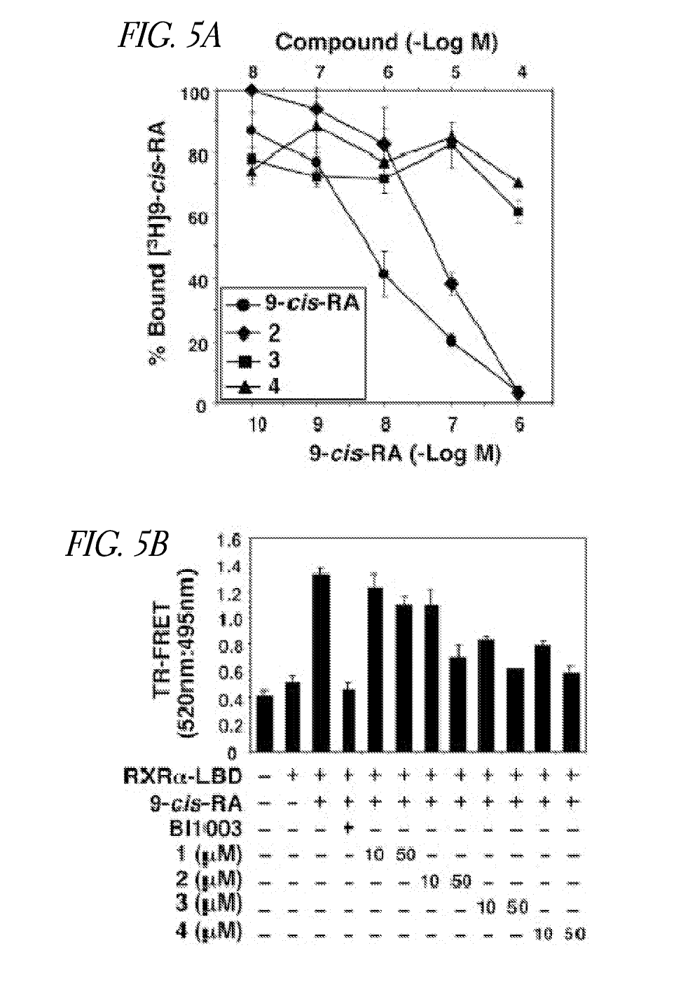

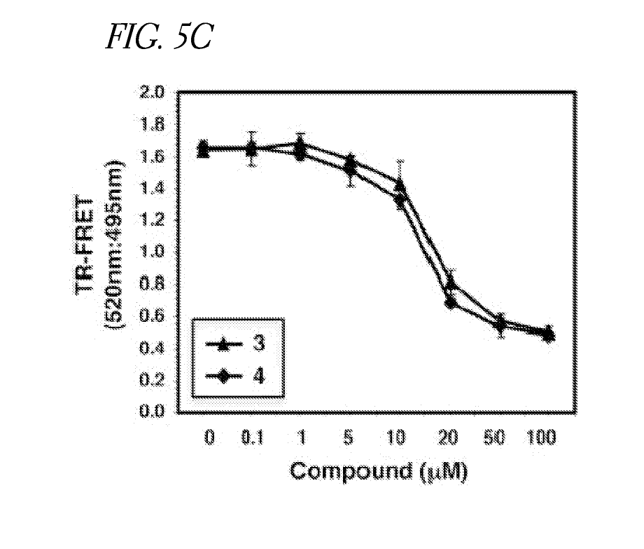

According to current understanding of the mechanism by which ligands regulate the transcriptional activity of nuclear receptors, K-8008 and K-8012 might bind to the canonical binding site, the LBP of RXRα, acting as conventional antagonists. Thus, binding to the LBP of RXRα using the classical radioligand competition binding assay was examined (Zhou et al., 2010). Unlike 9-cis-RA and K-80003 that competed well with [³H] 9-cis-RA for binding to the LBP of RXRα, K-8008 and K-8012 failed to replace [³H]9-cis-RA for its binding to the RXRα LBP (FIG. 5A).

Results of the [³H]9-cis-RA binding competition assay demonstrated that K-8008 and K-8012 did not bind to the canonical binding site, suggesting a different binding mechanism. Other than the classical LBP, recent structural and functional studies have revealed the existence of distinct small molecule binding sites on the surface of the LBD of nuclear receptors (Buzon et al., 2012; Moore et ah, 2010).

Whether K-8008 and K-8012 could bind to an alternative surface binding site was examined by using the time-resolved fluorescence resonance energy transfer (TR-FRET) RXRα co-activator peptide competition assay. The results showed that both compounds could inhibit 9-cis-RA-induced interaction of RXRα LBD with its coactivator peptide (FIG. 5B). The inhibitory effect of K-8008 and K-8012 was much stronger than Sulindac, with IC50 values of 16.8 μM and 14.5 μM, respectively (FIG. 5C), which correlated well with their inhibition of 9-m-RA-induced RXRα transactivation (FIG. 2D). Taken together, K-8008 and K-8012 might act as RXRα antagonists by binding to a novel RXRα surface site, leading to inhibition of coactivator binding.

### Example 4

### K-8008 and K-8012 Bind to a Tetrameric Structure of the RXRα LBD

To gain direct and structural understanding of the binding of K-8008 or K-8012 to RXRα, crystallographic studies of these ligands bound to the RXRα LBD were performed. Crystals of protein-ligand complexes were obtained using co-crystallization method. The structures of RXRα LBD in complex with K-8008 and K-8012 were determined to the resolution of 2.0 Å and 2.2 Å, respectively. Both protein/ligand complexes crystallized as tetrameric oligomers in the space group of P2₁ with similar unit cell parameters and the molecular replacement method was used to obtain the initial phasing by using the published RXRα structure, PDB code 1G1U. Statistics of structure refinement and data collection is summarized in Table 1.

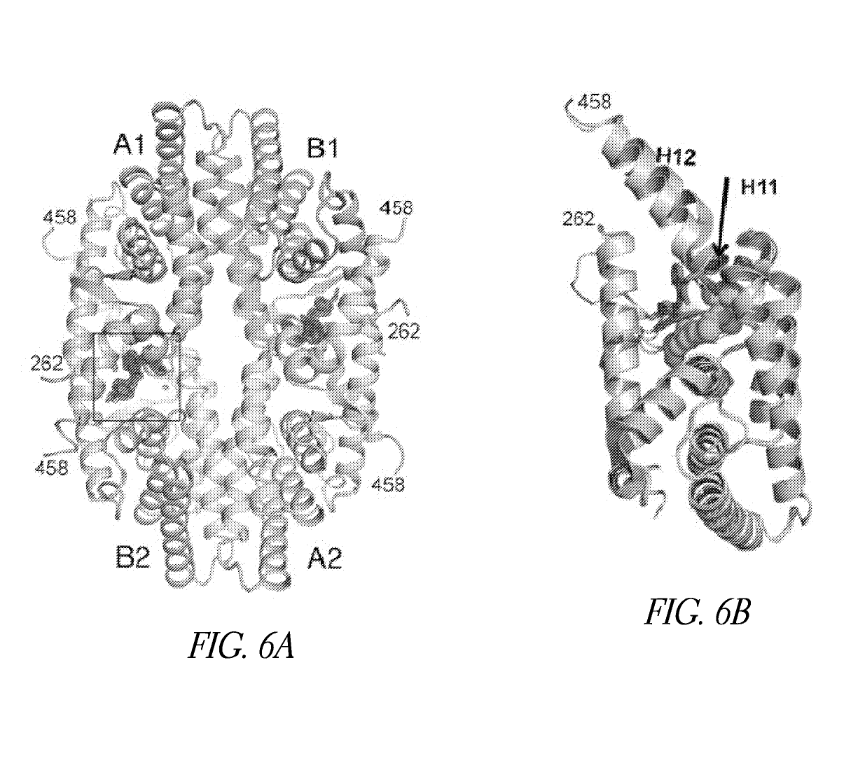

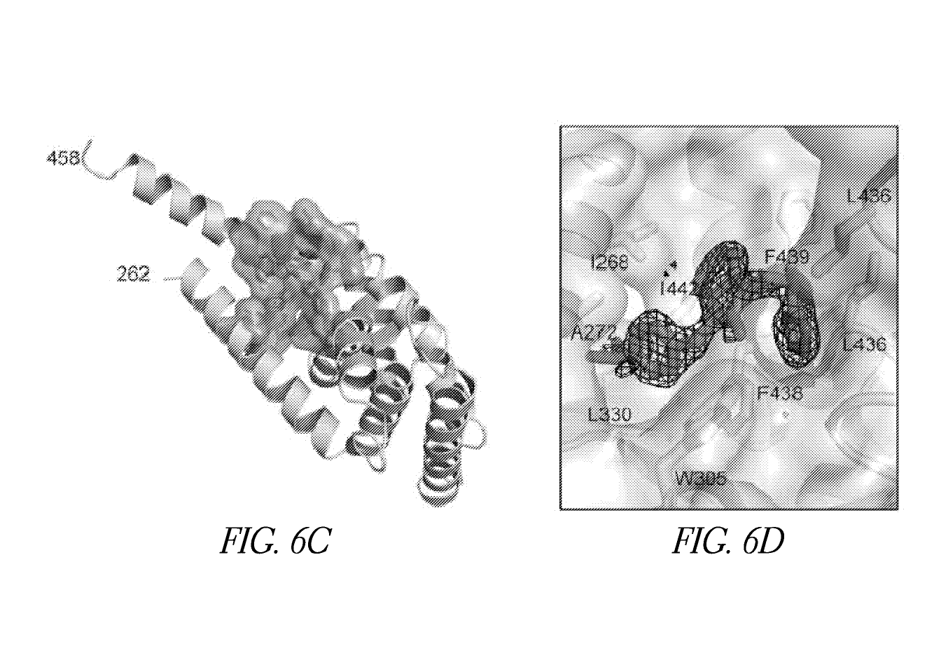

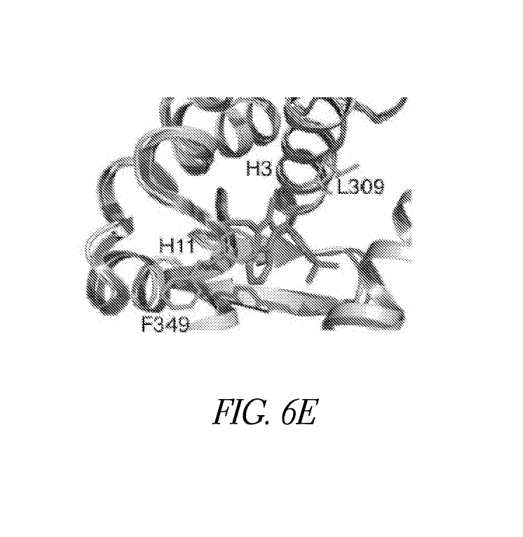

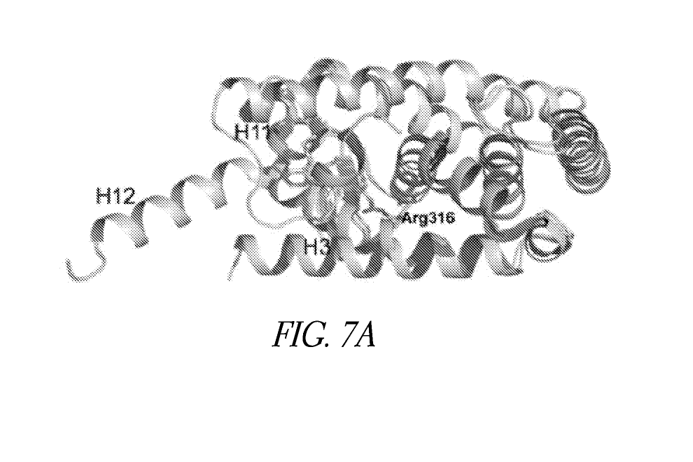

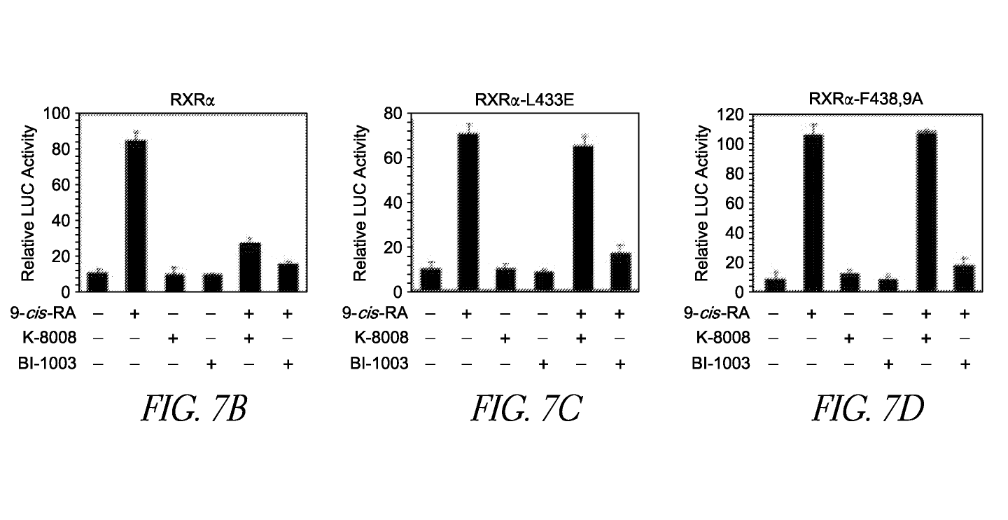

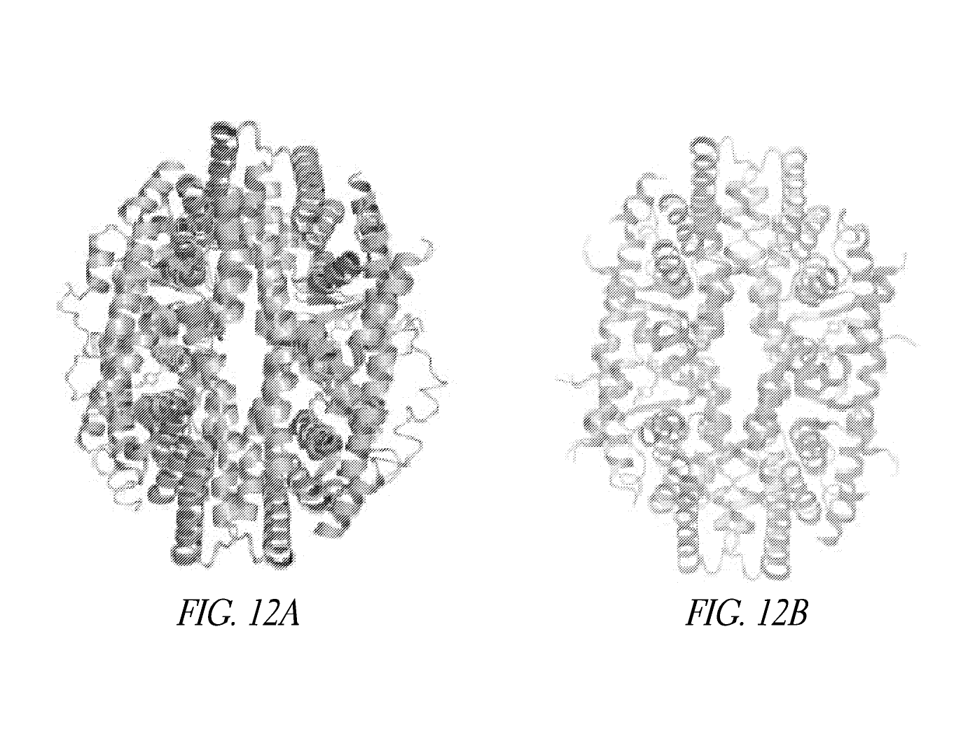

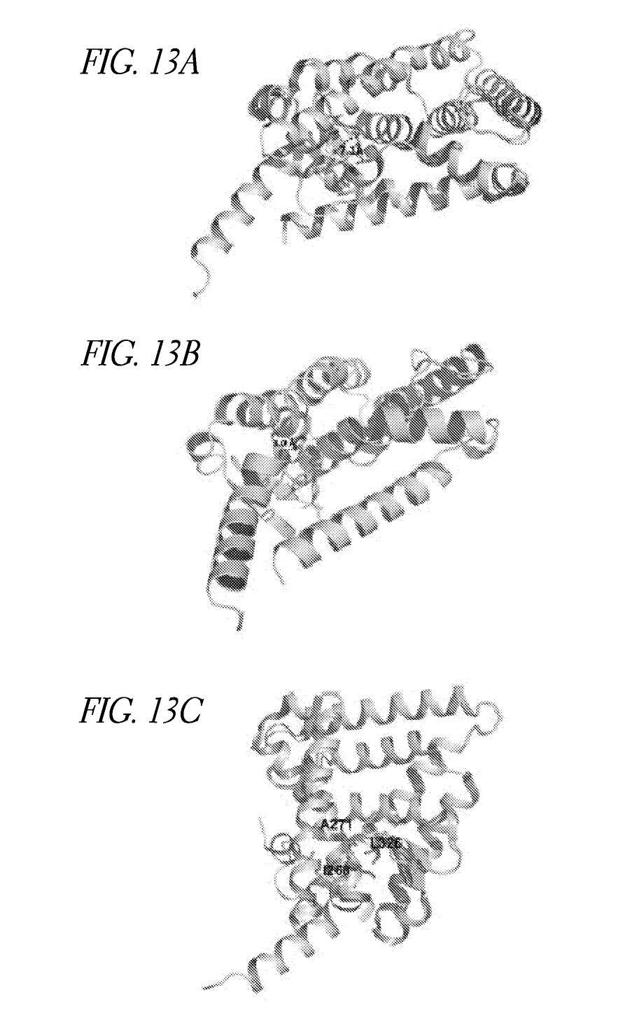

The crystal structure of the RXRα LBD in complex with the K-8008 exists as noncrystallograohic homo-tetramer similar to the reported apo homotetramer (Gampe et al., 2000), in which 2 homodimers pack in a bottom-to-bottom manner (FIG. 6A and FIG. 12). Superposition of this crystal structure with the published apo structure (PDB code 1G1U) shows that the corresponding monomers have almost identical fold with small shift found in the orientation of H12 in the monomer where a K-8008 molecule is bound (FIG. 6B). N-terminal residues, from 223 to 260, were found to be disordered and undetermined in the complex structures, though residues from 231 to 260 were defined in the Apo structure. In a tetramer, 2 modulator molecules were found to bind to one homotetramer, with a binding stoichiometric ratio of 1:2 between ligand and protein, as one ligand molecule binds only to one monomer within a dimer (FIG. 6A). K-8008 binds to a region that is close to the dimer-dimer interface, making interaction primarily with one monomer of the dimer and some interaction with one monomer of the other dimer. The structure of RXRα LBD in complex with K-8012 is very similar to that of RXRα LBD in complex with K-8008 (FIG. 13).

### Example 5

### K-8008 and K-8012 Bind to a New Hydrophobic Site of RXRα

Both K-8008 and K-8012 bind to a hydrophobic region of LBD near the entry and the edge of the cognate LBP. This region does not overlap with the binding region of 9-cis-RA (FIG. 6C), which suggests why both compounds failed to compete with the binding of 9-cis-RA (FIG. 5A). This hydrophobic region is made of side chains primarily from one monomer: Ala271 and Ala272 from H3, Trp305 and Leu309 from H5, Leu326 and Leu330 from the beta-turn, Leu433 from H10, Leu436 from L10-11, Phe437, Phe438, Ile442 and Gly443 from HI 1 of chain B2, and Leu436 from L10-11 of chain A1 (FIG. 6C). With respect to the monomer of RXRα LBD, this region is located on the surface of the RXRα monomer molecule. However, in the tetramer structure, this region is buried. K-8008 makes both hydrophobic interaction and polar interaction with the protein. The negatively charged tetrazole of the ligand sits on the top of the N-terminal end of HI 1, making charge-diploe interaction (FIG. 6B). The lipophilic part of the ligand makes hydrophobic interaction with side chains of Ile268, Ala271, Trp305. Leu436, Phe438, Phe439 and Ile442 from chain B2 and Leu436 from chain A1 (FIGS. 6D and 6A). Binding of K-8008 does not induce much significant changes in the surrounding side chains except for the side chains of Phe439 and Leu309. Side chain of Ph439 swings out to make room for the ligand to bind and side chain of Leu309 rearranges to make better VDW contact with the protein (FIG. 6E).

At least two new compounds, K-8008 and K-8012 are identified herein, which showed potent tRXRα inhibitory effects through a novel and unique binding mechanism. The results described herein, demonstrated that K-8008 and K-8012 were more effective than Sulindac in inhibiting RXRα transactivation (FIG. 2A). Sulindac binds to RXRα with an IC50 of 82.9 μM based on the classical ligand competition assays (Zhou et al., 2010). K-8008 and K-8012 could antagonize 9-cis-RA-induced transactivation and inhibit coactivator peptide binding to RXRα with IC50 value of around 10 μM. Consistently, K-8008 and K-8012 showed improved activity than Sulindac in inhibiting AKT activation and inducing apoptosis. About 100 μM of Sulindac is normally used to achieve its anti-cancer effects (Weggen et al., 2001; Yamamoto et al., 1999; Zhang et al., 2000), whereas 10 to 50 μM of K-8008 and K-8012 were able to inhibit AKT activation and induce apoptosis of cancer cells. Furthermore, K-8008 showed potent inhibitory effect on the growth of tumor cells in animals without apparent toxicity. Inhibition of AKT activation and induction of apoptosis by K-8008 and K-8012 were RXRα dependent, likely due to their inhibition of the interaction between tRXRcx and p85α.

Based on the principle of bioisosteric replacement (Matta et al., 2010), it was anticipated that tetrazole group acted like the carboxylate group, a common motif found in most of the cognate RXR ligands, which interacts with Arg316 in the LBP, and therefore both K-8008 and K-8012 would compete 9-cis-RA for binding. Unexpectedly, both K-8008 and K-8012, unlike Sulindac and K-80003, failed to compete with 9-cis-RA for binding to the LBP, demonstrating that they exert their antagonist effect through a different binding mechanism from Sulindac and K-80003. The structure analysis confirmed that the tetrazole group of K-8008 and K-8012 binds to a region away from Arg316 and it anchors to the RXRα protein by sitting atop the N-terminus of helix 11, forming the charge-helix dipole interaction. This charge-dipole interaction may also function to stabilize the orientation and conformation of H11 as in most of the cases ligand binding to the cognate LBP induces the conformation change and reorientation of HI 1 (Egea et al., 2000; Sato et al., 2010; Zhang et al., 2011). Furthermore, the structural results show that the compounds bind to a region that doesn't overlap with the 9-cA-RA binding space, offering a structural explanation for the inability of K-8008 and K-8012 to compete with 9-cis-RA for RXRα binding.

The crystal structures revealed that K-8008 and K-8012 bind to a RXRα LBD tetramer structure through a novel hydrophobic region that is located on the surface of a monomer and near the dimer-dimer interface in the tetramer. Unlike the binding of other ligands, the binding of K-8008 does not change the shape of the apo RXRα LBP. In addition, K-8008 interacts with monomers of each dimer in the tetramer, contributing to the dimer-dimer interaction. Taken together, K-8008 or K-8012 binding may help to stabilize the tetramer. Stabilizing the tetrameric state of RXRα through ligand binding may have important implication for the regulation of the nongenomic biological activities of RXRα.

K-8008 and K-8012 bind to a surface hydrophobic site and display weak antagonist effect. However, the therapeutic relevance of targeting the RXRα through this new binding site is evidenced by the observation that both K-8008 and K-8012 could inhibit tRXRα activities in cancer cells in vitro and tumor growth in animals (FIG. 4). Consistently, it was observed that administration of K-8008 at the dose that effectively inhibited the growth of tumor cells did not show any apparent toxicity to animals (FIG. 4). Thus, although showing a relatively weak binding to RXRα, these new compounds can be clinically relevant.

### Experimental Procedures

### Compound Synthesis

K-8008 and K-8012 were synthesized using scheme of FIG. 1B. See Supporting Information for details.

### Cell Culture and Transfection

PC3 prostate cancer, ZR-75-1 breast cancer and HeLa cervical cancer cells were grown in RPMI1640, CV-1 African green monkey kidney cells, HCT-16 colon cancer, A549 lung cancer cells were cultured in DMEM containing 10% fetal bovine serum. The cells were maintained at 5% CO₂ at 37° C. Subconfluent cells with exponential growth were used throughout the experiments. Cell transfections were carried out by using Lipofectamine 2000 (Invitrogen) according to the instructions of the manufacturer. Myc-RXRα-D80 and Flag-p85α expression vectors as well as RXRα siRNA were described (Zhou et al., 2010).

### CAT Assay

(TREpal)₂-tk-CAT (100 ng), (β-galactosidase (100 ng) and RXRα (20 ng) were transiently transfected into CV-1 cells (Zhang et al., 1992). Cells were then treated with or without 9-cis-RA (10⁻⁷ M) in the presence or absence of increasing concentrations of compounds for an additional 24 h. Cells were harvested and assayed for CAT and (3-gal activity. To normalize for transfection efficiency, CAT activities were corrected to (3-gal activities.

### Mammalian One Hybrid

HCT-116 cells seeded in 24-well plates were transiently transfected with 50 ng pG5luc, 25 ng pBind-RXRα-LBD. Twenty-four hours after transfection, the medium was replaced by medium containing other compounds, such as K-8008 or K-8012, and/or 9-cis-RA. Cells were washed, lysed and assayed by using the Dual-Luciferase Reporter Assay System (Promega). Transfection efficiency was normalized to Renilla luciferase activity.

### Protein Expression and Purification

The human RXRα LBD (residues Thr223 to Thr462) was cloned as an N-tenninal histidine-tagged fusion protein in pET 15b expression vector and overproduced in Escherichia coli BL21 strain. Briefly, cells were harvested and sonicated, and the extract was incubated with the His60 Ni Superflow resin. The protein-resin complexes were washed and eluted. The eluent was collected and concentrated to 5 mg/mL. For crystallization experiment, the His tag was cleaved by bovine thrombin (Sigma) and removed on the Resource-Q column (GE), using 0.1-1.0 M NaCl gradient and the TrisCl pH 8.0 buffer. The additional purification was done by the gel filtration on a Superdex-200 2660 column (GE) pre-equilibrated with the 75 mM NaCl, 20 mM Tris-Cl buffer (pH 8.0).

### Ligand-Binding Competition Assay

The His-tagged human RXRα-LBD(223-462) was incubated in tubes with unlabeled 9-cis-RA or different concentrations of compounds in 200 μL binding buffer [0.15 M KCl, 10 mM Tris HCl (pH7.4), 8% glycerol, and 0.5% CHAPS detergent] at 4° C. for 1 h. [³H]-9-cis-RA was added to the tubes to final concentration of 7.5 nM and final volume of 300 μL and incubated overnight at 4° C. The RXRα-LBD was captured by nickel-coated beads. Bound [³H]-9-cis-RA was quantitated by liquid scintillation counting.

### TR-FRET Retinoic X Receptor Alpha Coactivator Assay

Invitrogen's LanthsScreen TR-FRET RXRα Coactivator Assay was conducted according to the manufacture's protocol. The TR-FRET ratio was calculated by dividing the emission signal at 520 nm by the emission signal at 495 nm.

### MTT Assay

Confluent cells cultured in 96-well dishes were treated with various concentrations of compounds for 48 h. The cells were then incubated with 2 mg/mL (3-(4,5-Dimethylthiazol-2-yl)-2,5-diphenyltetrazolium bromide (MTT) for 4 h at 37° C. MTT solution was then aspirated and formazan in cells was instantly dissolved by addition of 150 μL DMSO each well. Absorbance was measured at 570 nm.

### Western Blotting

Cells were lysed and equal amounts of the lysates were electrophoresed on 10% SDS-PAGE gels and transferred onto PVDF membranes (Millipore). The membranes were blocked with 5% skimmed milk in TBST [50 mM Tris-HCl (pH7.4), 150 mM NaCl and 0.1% Tween20] for 1 h, then incubated with primary antibodies and secondary antibodies and detected using ECL system (Thermo). The dilutions of the primary antibodies were anti-RXRα (AN 197, Santa Cruz) in 1:1000, anti-PARP(H-250. Santa Cruz) in 1:3000, anti-p85α (Millipore) in 1:1000, anti-p-AKT (D9E, Cell Signaling Technology) in 1:1000, anti-AKT 1/2/3 (H-136, Santa Cruz) in 1:1000, anti-β-actin (Sigma) in 1:5000, anti-c-myc (9E10, Santa Cruz), anti-Flag (FI 804, Sigma).

### Co-Immunoprecipitation Assay

Cells were harvested and lysed in buffer containing 50 mM Hepes-NaOH (pH7.5), 2.5 mM EDTA, 100 mM NaCl, 0.5% NP40, and 10% glycerol, with 1 mM DTT and proteinase inhibitor cocktail. Immunoprecipitation was performed as described (Zhou et al., 2010).

### HepG2 Xenografts

Nude mice (BALB/c, SPF grade. 16-18 g, 4-5-week old) were housed at 28° C. in a laminar flow under sterilized conditions. Mice were injected subcutaneously with 100 μL HepG2 cells (2×10⁻⁶). For drug treatment, mice (n=6) were treated intraperitoneally after 7 days of transplantation with K-8008 (20 mg/kg), K-80003 (20 mg/kg) or vehicle (tween-80) once a day. Body weight and tumor size were measured every 3 days. Mice were sacrificed after 12-day drug treatment and the tumors removed for various assessments.

### Histology and Apoptosis Analysis

Paraffin wax embedded tumors were cut into 5 |iM-thick sections. These sections were deparaffinized and stained with hematoxylin and eosin (H&E) according to the standard protocol. Tumor sections of HepG2 xenografts were also stained with TUNEL for assessing spontaneous apoptosis according to the manufacturer's instructions (In situ Cell Death Detection Kit; Roche). The images were taken under a fluorescent microscope (Carl Zeiss).

### Crystallization and Structure Solution of the RXR LBD-Ligand Complexes

Both crystal structures have the space group P2₁ and similar unit cell parameters, however, axes b and c have been replaced in them (see Table 1). This difference was not induced by differences in ligands, because K-8008 co-crystals were also obtained in same unit cell as K-8012 co-crystals (data are not presented). More likely, the crystal packing changed due to the presence of different salt additives (Mg Formate in case of the K-8008 co-crystal and Na Acetate in case of the K-8012 co-crystal) during the crystallization. Nevertheless, the change of the unit cell did not affect protein structures significantly. They both crystallized as noncrystallographic tetramers with molecular symmetry P222, and the rms deviation between their 760 Ca atoms (out of 788) is 0.36 Å, which is comparable with the overall error of the structures (0.3 Å). In both structures, the chains A and D as well as chains B and C have a very high degree of pairwise similarity. Thus, in the RXRα LBD/K-8008 structure, the rms deviations between 193 out of 197 Ca atoms of the chains A and D was 0.24 Å (0.22 Å for the chains B and C). The rms deviation between chains A and B (as well as A and C) was 0.6 Å (for the same group of Ca atoms). Several N- and C-terminal residues of both structures are disordered. Thus, the electron density is present for residues 261-457 of all four chains of the RXRα LBD/K-8008 structure. In the RXRα LBD/K-8012-binding structure, however, the 10 additional residues at the N-termini of the chains B and C (residues 231-241) are also ordered. Interestingly, in the crystal structure of RXRα LBD complexed with an inactive retinoic acid isomer (PDB entry 1G5Y), the entire region 231-458 of all four chains is ordered, even though its unit cell parameters (a, b, c=51.0, 99.7, 96.3 Å β=96.70) are very close to those of the K-8008 co-crystal.

The initial crystallization conditions were determined using the sitting-drop vapor-diffusion method and the crystallization screens Index and PEG-Ion (Hampton research). Other crystallization chemicals were from Hampton research and Sigma. The data were collected from crystals grown in sitting drops of the 96-well Intelli-Plates (ARI) by the vapor diffusion method. 0.2 pi of the protein-ligand complex containing 0.37 mM of RXR LBD, 0.5-0.7 mM of a ligand, 100 mM NaCl and 20 mM Tris-Cl buffer (pH 8.0) were mixed with 0.2 μl of the well solution (20% PEG3330 and 0.2M Magnesium Formate for the K-8008 complex or 0.2 M Na Acetate for the K-8012 complex) and incubated at 20° C. The first crystals appeared in 5-10 days and grew within same amount of time into 0.2×0.2×0.05 mm plates. The crystals were flash-frozen against the well solution containing 20% PEG400 as a cryoprotectant. The diffraction data were collected from the cryo-cooled crystals (@100° K) at the beamline BL11-2 of SSRL and processed using the program suits XDS (Kabsch, 2010) and ccp4i (Collaborative Computational Project, 1994).

The structures were solved by the molecular replacement program Phaser (McCoy et al., 2007) using pdb entry 1G1U as an initial model. The model rebuilding and refinement were done with Coot (Emsley and Cowtan, 2004) and the program suit Phenix (Adams et al., 2010). The initial models and parameter files for the ligands were prepared by eLBOW of Phenix. The data collection and refinement statistics are presented in Table 1.

TABLE 1

| | RXR LBD co-crystalized with- | | | |
|---|---|---|---|---|
| | K-8008 | | K-8012 | |
| Space Group | P2₁ | | P2₁ | |
| Unit Cell a, b, c/Å, β | 51.0 99.3 | | 46.6 98.8 | |
| | 94.0 98.7° ° | | 110.6 99.0 | |
| Resolution/Å | 68-2.0 | | 37-2.1 | |
| (outer shell) | (2.08-2.03) | | (2.17-2.11) | |
| Unique reflections | 58010 | | 48947 | |
| collected | | | | |
| Completeness (%) | 97 | (92) | 86 | (50) |
| Average Redundancy | 4.3 | (3.5) | 3.6 | (3.4) |
| <I/d(I)> | 8.6 | (1.0) | 8.1 | (1.6) |
| CC(1/2) | 0.988 | (0.86) | 0.997 | (0.71) |
| Rₘₑₐₛ | 0.16 | (1.4) | 0.079 | (0.80) |
| Refinements statistics: | | | | |
| Resolution range (Å) | 50-2.0 | | 37-2.2 | |
| No reflections work set | 57918 | (3719) | 44889 | (4288) |
| (R_{FREE} set) | | | | |
| R_{WORK} (R_{FREE}) | 0.199 | (0.237) | 0.199 | (0.247) |
| RMS Deviations: | | | | |
| bond lengths (Å) | 0.003 | | 0.003 | |
| bond angles (Å) | 0.74 | | 0.76 | |
| Ramachandran plot (%): | | | | |
| Favored by | 97.6 | | 98.2 | |
| MolProbity (%) | | | | |
| Outliers by | 0.0 | | 0.0 | |
| MolProbity (%) | | | | |
| Coordinate errors, | 0.30 | | 0.29 | |
| estimated by | | | | |
| Phenix (Å) | | | | |
| No. of protein residues | 788 | (976) | 810 | (976) |
| observed (present) | | | | |
| No. of ligand residues | 2 | | 2 | |
| No. of Water molecules | 652 | | 280 | |
| Temperature factors (A²): | | | | |

| | | |
|---|---|---|
| overall | 21.4 | 50.4 |
| protein | 20.8 | 50.6 |
| ligands | 29.7 | 53.4 |
| solvent | 26.5 | 45.6 |
| from Wilson B plot | 28.1 | 40.3 |

### Data Analyses

Data were expressed as means±SD from three or more experiments. Statistical analysis was performed using Student's t test. Differences were considered statistically significant with P<0.05.

### Accession Numbers

The coordinates for the crystal structures of RXRα LBD in complex with K-8008 or K-8012 were deposited with the Protein Data Bank under ID codes 4N8R and 4N5G, respectively.

### REFERENCES

Adams, P. D., Afonine, P. V., Bunkoczi, G., Chen, V. B., Davis, I. W., Echols, N., Headd, J. J., Hung, L. W., Kapral, G. J., Grosse-Kunstleve, R. W., et al. (2010). PHENIX: a comprehensive Python-based system for macromolecular structure solution. Acta crystallographica Section D, Biological crystallography 66, 213-221.
Arnold, L. A., Estebanez-Perpina, E., Togashi, M., Jouravel, N., Shelat, A., McReynolds, A. C., Mar, E., Nguyen, P., Baxter. J. D., Fletterick, R. J., et al. (2005). Discovery of Small Molecule Inhibitors of the Interaction of the Thyroid Hormone Receptor with Transcriptional Coregulators. Journal of Biological Chemistry 280, 43048-43055.
Balkwiil, F. (2009). Tumour necrosis factor and cancer. Nature reviews Cancer 9, 361-371.
Bourguet, W., Ruff, M., Bonnier, D., Granger, F., Boeglin, M., Chambon, P., Moras, D., and Gronemeyer, H. (1995). Purification, functional characterization, and crystallization of the ligand binding domain of the retinoid X receptor. Protein expression and purification 6, 604-608.
Bushue, N., and Wan, Y.-J. Y. (2010). Retinoid pathway and cancer therapeutics. Advanced Drug Delivery Reviews 62, 1285-1298.
Buzon, V., Carbo, L. R., Estruch, S. B., Fletterick, R. J., and Estebanez-Perpina, E. (2012). A conserved surface on the ligand binding domain of nuclear receptors for allosteric control. Molecular and cellular endocrinology 348, 394-402.
Caboni, L., Kinsella, G. K., Blanco, F., Fayne, D., Jagoe, W. N., Carr, M., Williams, D. C., Meegan, M. J., and Lloyd, D. G. (2012a). “True” antiandrogens-selective non-ligand-binding pocket disruptors of androgen receptor-coactivator interactions: novel tools for prostate cancer. J Med Chem 55, 1635-1644. Caboni, L., Kinsella, G. K., Blanco, F., Fayne, D., Jagoe, W. N., Carr, M., Williams, D. C., Meegan, M. J., and Lloyd, D. G. (2012b). “True” Antiandrogens-Selective Non-Ligand-Binding Pocket Disruptors of Androgen Receptor-Coactivator Interactions: Novel Tools for Prostate Cancer, J Med Chem 55, 1635-1644.
Cao, X., Liu, W., Lin, F., Li, H., Kolluri, S. K., Lin, B., Han, Y.-H., Dawson, M. I., and Zhang, X.-k. (2004). Retinoid X receptor regulates Nur77/TR3-dependent apoptosis by modulating its nuclear export and mitochondrial targeting. Mol Cell Biol 24, 9705-9725.
Casas, F., Daury, L., Grandemange, S., Busson, M., Seyer, P., Hatier, R., Carazo, A., Cabello, G., and Wrutniak-Cabello. C. (2003). Endocrine regulation of mitochondrial activity: involvement of truncated RXRαlpha and c-Erb Aalphal proteins. FASEB J 17, 426-436.
Chen, Z.-P., Iyer, J., Bourguet, W., Held, P., Mioskowski, C., Lebeau, L., Noy, N., Chambon, P., and Gronemeyer, H. (1998). Ligand- and DNA-induced dissociation of RXR tetramers. J Mol Biol 275, 55-65.
Collaborative Computational Project. N. (1994). The CCP4 suite: programs for protein crystallography. Acta crystallographica Section D, Biological crystallography 50, 760-763.
Davies, N. M., and Watson, M. S. (1997). Clinical Pharmacokinetics of Sulindac: A Dynamic Old Drug. Clinical Pharmacokinetics 32, 437-459.
Dawson, M. I., and Zhang, X. K. (2002). Discovery and design of retinoic acid receptor and retinoid X receptor class- and subtype-selective synthetic analogs of all-trans-retinoic acid and 9-cis-retinoic acid. Current medicinal chemistry 9, 623-637.
de Lera, A. R., Bourguet, W., Altucci, L., and Gronemeyer, H. (2007). Design of selective nuclear receptor modulators: RAR and RXR as a case study. Nature reviews Drug discovery 6, 811-820.
Dufour, J. M., and Kim, K. H. (1999). Cellular and subcellular localization of six retinoid receptors in rat testis during postnatal development: identification of potential heterodimeric receptors. Biol Reprod 61, 1300-1308.
Duggan, D. E., Hooke, K. F., and Hwang, S. S. (1980). Kinetics of the tissue distributions of sulindac and metabolites. Relevance to sites and rates of bioactivation. Drug Metabolism and Disposition 8, 241-246. Egea, P. F., Mitschler, A., Rochel, N., Ruff, M., Chambon, P., and Moras, D. (2000). Crystal structure of the human RXR[alpha] ligand-binding domain bound to its natural ligand: 9-cis retinoic acid. EMBO J 79, 2592-2601.
Emsley, P., and Cowtan, K. (2004). Coot: model-building tools for molecular graphics. Acta crystallographica Section D, Biological crystallography 60, 2126-2132.
Fukunaka, K., Saito, T., Wataba, K., Ashihara, K., Ito, E., and Kudo, R. (2001). Changes in expression and subcellular localization of nuclear retinoic acid receptors in human endometrial epithelium during the menstrual cycle. Mol Hum Reprod 7, 437-446.
Gampe, R. T., Montana, V. G., Lambert, M. H., Wisely, G. B., Milbum, M. V., and Xu, H. E. (2000). Structural basis for autorepression of retinoid X receptor by tetramer formation and the AF-2 helix. Genes Dev 14, 2229-2241.
Germain, P., Chambon, P., Eichele, G., Evans, R. M., Lazar, M. A., Leid, M., De Lera, A. R., Lotan, R., Mangelsdorf, D. J., and Gronemeyer, H. (2006). International Union of Pharmacology. LXIII. Retinoid X Receptors. Pharmacological Reviews 58, 760-772.
Gunther, J. R., Moore, T. W., Collins, M. L., and Katzenellenbogen, J. A. (2008). Amphipathic Benzenes Are Designed Inhibitors of the Estrogen Receptor a/Steroid Receptor Coactivator Interaction. ACS Chem Biol 3, 282-286.
Herr, R. J. (2002). 5-Substituted-1H-tetrazoles as carboxylic acid isosteres: medicinal chemistry and synthetic methods. Bioorg Med Chem 10, 3379-3393.
Hwang, J. Y., Arnold, L. A., Zhu, F., Kosinski, A., Mangano, T. J., Setola, V., Roth, B. L., and Guy, R. K. (2009). Improvement of Pharmacological Properties of Irreversible Thyroid Receptor Coactivator Binding Inhibitors. J Med Chem 52, 3892-3901.
Kabsch, W. (2010). Xds. Acta crystallographica Section D, Biological crystallography 66, 125-132. Kersten, S., Dong, D., Lee, W., Reczek, P. R., and Noy, N. (1998). Auto-silencing by the retinoid X receptor. J Mol Biol 284, 21-32.
Kersten, S., Kelleher, D., Chambon, P., Gronemeyer, H., and Noy, N. (1995). Retinoid X receptor alpha forms tetramers in solution. Proceedings of the National Academy of Sciences 92, 8645-8649.
Kojetin, D. J., Burris, T. P., Jensen, E. V., and Khan, S. A. (2008). Implications of the binding of tamoxifen to the coactivator recognition site of the estrogen receptor. Endocrine-Related Cancer 15, 851-870.
Kolluri, S. K., Corr. M., James, S. Y., Bemasconi, M., Lu, D., Liu, W., Cottam, H. B., Leoni, L. M., Carson, D. A., and Zhang, X.-k. (2005). The R-enantiomer of the nonsteroidal antiinflammatory drug etodolac binds retinoid X receptor and induces tumor-selective apoptosis. Proceedings of the National Academy of Sciences of the United States of America 102, 2525-2530.
Lazebnik, Y. A., Kaufmann, S. H., Desnoyers, S., Poirier, G. G., and Eamshaw, W. C. (1994). Cleavage of poly(ADP-ribose) polymerase by a proteinase with properties like ICE. Nature 371, 346-347.
Lu, J., Dawson, M. I., Hu, Q. Y., Xia, Z., Dambacher, J. D., Ye, M., Zhang, X.-K., and Li, E. (2009). The effect of antagonists on the conformational exchange of the retinoid X receptor alpha ligand-binding domain. Magnetic Resonance in Chemistry 47, 1071-1080.
Matta, C. F., Arabi, A. A., and Weaver, D. F. (2010). The bioisosteric similarity of the tetrazole and carboxylate anions: Clues from the topologies of the electrostatic potential and of the electron density. EurJMed Chem 45, 1868-1872.
McCoy, A. J., Grosse-Kunstleve, R. W., Adams, P. D., Winn, M. D., Storoni, L. C., and Read, R. J. (2007). Phaser crystallographic software. Journal of applied crystallography 40, 658-674.
Mita, Y., Dodo, K., Noguchi-Yachide, T., Miyachi, H., Makishima, M., Hashimoto, Y., and Ishikawa, M. (2010). LXXLL peptide mimetics as inhibitors of the interaction of vitamin D receptor with coactivators. Bioorganic &amp; Medicinal Chemistry Letters 20, 1712-1717.
Mizwicki, M. T., Keidel, D., Bula, C. M., Bishop, J. E., Zanello, L. P., Wurtz, J. M., Moras, D., and Norman, A. W. (2004). Identification of an alternative ligand-binding pocket in the nuclear vitamin D receptor and its functional importance in lalpha, 25(OH)2-vitamin D3 signaling. Proc Natl Acad Sci USA 101, 12876-12881.
Moore, T. W., Mayne, C. G., and Katzenellenbogen, J. A. (2010). Minireview: Not Picking Pockets: Nuclear Receptor Alternate-Site Modulators (NRAMs). Mol Endocrinol 24, 683-695.
Peet, D. J., Doyle, D. F., Corey, D. R., and Mangelsdorf, D. J. (1998). Engineering novel specificities for ligand-activated transcription in the nuclear hormone receptor RXR. Chemistry & biology 5, 13-21. Rodriguez, A. L., Tamrazi. A., Collins, M. L., and Katzenellenbogen, J. A. (2003). Design, Synthesis, and in Vitro Biological Evaluation of Small Molecule Inhibitors of Estrogen Receptor a Coactivator Binding. J Med Chem 47, 600-611.
Sato, Y., Ramalanjaona, N., Huet, T., Potier, N., Osz, J., Antony, P., Peluso-Iltis, C., Poussin-Courmontagne, P., Ennifar, E., Mely, Y., et al. (2010). The “Phantom Effect” of the Rexinoid LG 100754: structural and functional insights. PloS one 5, el5119.
Sun, A., Moore, T. W., Gunther, J. R., Kim, M. S., Rhoden, E., Du, Y., Fu, H., Snyder, J. P., and Katzenellenbogen, J. A. (2011), Discovering small-molecule estrogen receptor alpha/coactivator binding inhibitors: high-throughput screening, ligand development, and models for enhanced potency. ChemMedChem 6, 654-666.
Szanto, A., Narkar, V., Shen, Q., Uray, I. P., Davies, P. J. A., and Nagy, L. (2004). Retinoid X receptors: X-ploring their (patho)physiological functions. Cell Death Differ 11, S126-S143

The term “comprising” as used herein is synonymous with “including,” “containing,” or “characterized by,” and is inclusive or open-ended and does not exclude additional, unrecited elements or method steps.

The above description discloses several methods and materials of the present invention. This invention is susceptible to modifications in the methods and materials, as well as alterations in the fabrication methods and equipment. Such modifications will become apparent to those skilled in the art from a consideration of this disclosure or practice of the invention disclosed herein. Consequently, it is not intended that this invention be limited to the specific embodiments disclosed herein, but that it cover all modifications and alternatives coming within the true scope and spirit of the invention.

All references cited herein, including but not limited to published and unpublished applications, patents, and literature references, are incorporated herein by reference in their entirety and are hereby made a part of this specification. To the extent publications and patents or patent applications incorporated by reference contradict the disclosure contained in the specification, the specification is intended to supersede and/or take precedence over any such contradictory material.

What is claimed is:

1. A compound of Formula (I) having the structure: 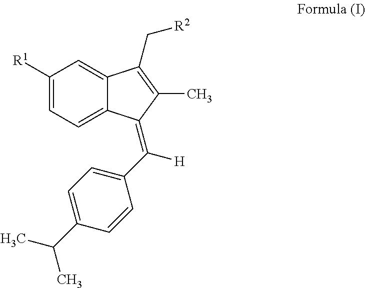
or tautomers thereof,
wherein:
R¹ is H or halogen; and
R² is C(═O)OH or

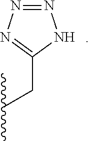 [SMILES: *Cc1nnn[nH]1]

2. The compound of claim 1, wherein R¹ is H.

3. The compound of claim 1, wherein R¹ is F.

4. The compound of any one of claims 1-3, wherein R² is C(═O)OH.

5. The compound of any one of claims 1-3, wherein R² is

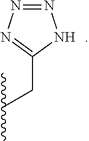 [SMILES: *Cc1nnn[nH]1]

6. The compound of claim 1 having the structure:

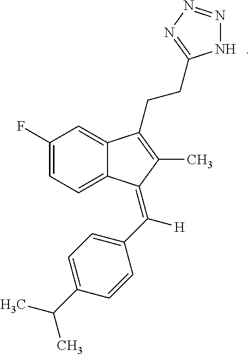 [SMILES: CC1=C(CCc2nnn[nH]2)c2cc(F)ccc2/C1=C\c1ccc(C(C)C)cc1]

7. The compound of claim 1 having the structure:

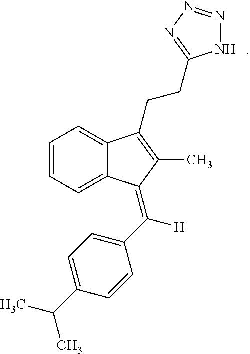 [SMILES: CC1=C(CCc2nnn[nH]2)c2ccccc2/C1=C\c1ccc(C(C)C)cc1]

8. A pharmaceutical composition comprising the compound of any one of claims 1-7 and a pharmaceutically acceptable excipient.

9. A method of inhibiting a tumor cell comprising contacting the tumor cell with the compound of any one of claims 1-7.

10. The method of claim 9, further comprising contacting the tumor cell with tumor necrosis factor-alpha (TNFα).

11. The method of any one of claims 9-10, wherein the tumor cell is selected from the group consisting of a lung tumor cell, a prostate tumor cell, a breast tumor cell, a colon tumor cell, an ovarian tumor cell and a liver tumor cell.

12. The method of any one of claims 9-10, wherein the tumor cell is selected from the group consisting of PC3, ZR-75-1, HeLa, HCT-116, A549, MB231, HepG2, and CV-1.

13. The method of any one of claims 9-12, wherein the tumor cell is in vivo.

14. The method of any one of claims 9-12, wherein the tumor cell is in vitro.

15. The method of any one of claims 9-14, wherein the tumor cell is mammalian.

16. The method of any one of claims 9-15, wherein the tumor cell is human.

17. A method of treating a tumor comprising administering to a subject in need thereof an effective amount of the pharmaceutical composition of claim 8.

18. The method of claim 17, further comprising administering an effective amount of tumor necrosis factor-alpha (TNFα) to the subject.

19. The method of any one claims 17-18, wherein the tumor is liver tumor.

20. The method of any one of claims 17-19, wherein the subject is mammalian.

21. The method of any one of claims 17-20, wherein the subject is human.

22. A kit comprising the compound of any one of claims 1-7 and a pharmaceutically acceptable excipient.

23. The kit of claim 22, further comprising an additional therapeutic agent.

24. The kit of claim 23, wherein the additional therapeutic agent comprises tumor necrosis factor-alpha (TNFα).

25. A method of designing a compound that binds to human RXRα protein comprising:
accessing data comprising the structure of at least the ligand binding domain (LBD) of human RXRα protein; and
modeling the binding of the compound to human RXRα protein using said data.

26. A method for identifying a compound for inhibiting growth of a tumor cell comprising the method of claim 25.

27. The method of any one of claims 25-26, wherein the modeling further comprises predicting the likelihood that the compound binds to a hydrophobic region of the LBD or RXRα protein that does not overlap with the binding site of 9-cis-retinoic acid.

28. The method of any one of claims 25-27, further comprising predicting that the compound does not change the conformation of the cognate ligand-binding pocket (LBP) of RXRα protein.

29. The method of any one of claims 25-28, wherein the compound is predicted to bind to a region of the LBD of RXRα protein comprising at least one residue selected from the group consisting of Ala271, Ala272, Trp305, Leu309, Leu326, Leu330, Leu433, Leu436, Phe437, Phe438, Ile442, Gly443, and Leu436.

30. The method of any one of claims 25-29, wherein the compound is designed de novo.

31. The method of any one of claims 25-29, wherein the compound is designed from a known chemical entity of fragment thereof.

32. The method of claim 31, wherein the chemical entity is the compound of any one of claims 1-7.

33. The method of claim 31, wherein the chemical entity is:

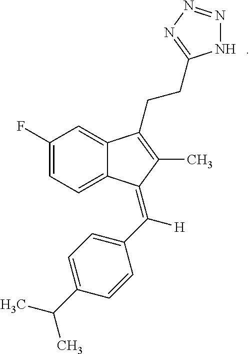 [SMILES: CC1=C(CCc2nnn[nH]2)c2cc(F)ccc2/C1=C\c1ccc(C(C)C)cc1]

34. The method of claim 31, wherein the chemical entity is: 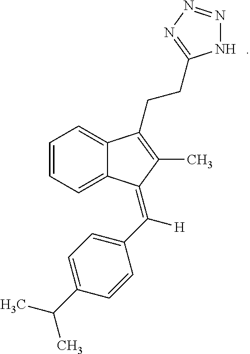 [SMILES: CC1=C(CCc2nnn[nH]2)c2ccccc2/C1=C\c1ccc(C(C)C)cc1]
